SEQUENCE LISTING (65 sequences)

SEQ ID NO: 1 - RNA, 19 nt, Artificial Sequence
  A SYNTHETIC SMALL INTERFERING NUCLEOTIDE
gguaacugcugggcauuua

SEQ ID NO: 2 - RNA, 19 nt, Artificial Sequence
  A SYNTHETIC SMALL INTERFERING NUCLEOTIDE
gguaccaguuuguuacuuu

SEQ ID NO: 3 - RNA, 19 nt, Artificial Sequence
  A SYNTHETIC SMALL INTERFERING NUCLEOTIDE
gcgcucaguuccagcuauu

SEQ ID NO: 4 - RNA, 19 nt, Artificial Sequence
  A SYNTHETIC SMALL INTERFERING NUCLEOTIDE
gaaaagacccgacgacaca

SEQ ID NO: 5 - RNA, 19 nt, Artificial Sequence
  A SYNTHETIC SMALL INTERFERING NUCLEOTIDE
gcacaaacaaaucagcuuu

SEQ ID NO: 6 - RNA, 19 nt, Artificial Sequence
  A SYNTHETIC SMALL INTERFERING NUCLEOTIDE
gcgcugucucccugaagaa

SEQ ID NO: 7 - RNA, 19 nt, Artificial Sequence
  A SYNTHETIC SMALL INTERFERING NUCLEOTIDE
gaaccgagcggccagaaca

SEQ ID NO: 8 - RNA, 19 nt, Artificial Sequence
  A SYNTHETIC SMALL INTERFERING NUCLEOTIDE
cgagcggccagaacaacaa

SEQ ID NO: 9 - RNA, 19 nt, Artificial Sequence
  A SYNTHETIC SMALL INTERFERING NUCLEOTIDE
ccgagcggccagaacaaca

SEQ ID NO: 10 - RNA, 19 nt, Artificial Sequence
  A SYNTHETIC SMALL INTERFERING NUCLEOTIDE
cagaagcacaaacaaauca

SEQ ID NO: 11 - RNA, 19 nt, Artificial Sequence
  A SYNTHETIC SMALL INTERFERING NUCLEOTIDE
aaccgagcggccagaacaa

SEQ ID NO: 12 - RNA, 19 nt, Artificial Sequence
  A SYNTHETIC SMALL INTERFERING NUCLEOTIDE
agcacaaacaaaucagcuu

SEQ ID NO: 13 - RNA, 19 nt, Artificial Sequence
  A SYNTHETIC SMALL INTERFERING NUCLEOTIDE
guaucaaccacgugucaag

SEQ ID NO: 14 - RNA, 19 nt, Artificial Sequence
  A SYNTHETIC SMALL INTERFERING NUCLEOTIDE
ccugcaggaugcugugacu

SEQ ID NO: 15 - RNA, 19 nt, Artificial Sequence
  A SYNTHETIC SMALL INTERFERING NUCLEOTIDE
cuguauuggacgagucuug

SEQ ID NO: 16 - RNA, 19 nt, Artificial Sequence
  A SYNTHETIC SMALL INTERFERING NUCLEOTIDE
cugaagaaccgagcggcca

SEQ ID NO: 17 - RNA, 19 nt, Artificial Sequence
  A SYNTHETIC SMALL INTERFERING NUCLEOTIDE
uaguaucaaccacguguca

SEQ ID NO: 18 - RNA, 19 nt, Artificial Sequence
  A SYNTHETIC SMALL INTERFERING NUCLEOTIDE
uuggacgagucuuggauuc

SEQ ID NO: 19 - RNA, 19 nt, Artificial Sequence
  A SYNTHETIC SMALL INTERFERING NUCLEOTIDE
gagucuuggauucgugaac

SEQ ID NO: 20 - RNA, 19 nt, Artificial Sequence
  A SYNTHETIC SMALL INTERFERING NUCLEOTIDE
caaaucagcuuuuaguauc

SEQ ID NO: 21 - RNA, 19 nt, Artificial Sequence
  A SYNTHETIC SMALL INTERFERING NUCLEOTIDE
agucuuggauucgugaaca

SEQ ID NO: 22 - RNA, 19 nt, Artificial Sequence
  A SYNTHETIC SMALL INTERFERING NUCLEOTIDE
ucaaccacgugucaaggca

SEQ ID NO: 23 - RNA, 19 nt, Artificial Sequence
  A SYNTHETIC SMALL INTERFERING NUCLEOTIDE
ugucaaggcaggucacguc

SEQ ID NO: 24 - RNA, 19 nt, Artificial Sequence
  A SYNTHETIC SMALL INTERFERING NUCLEOTIDE
auggugaggcugugggugc

SEQ ID NO: 25 - RNA, 19 nt, Artificial Sequence
  A SYNTHETIC SMALL INTERFERING NUCLEOTIDE
gagcggccagaacaacaaa

SEQ ID NO: 26 - RNA, 19 nt, Artificial Sequence
  A SYNTHETIC SMALL INTERFERING NUCLEOTIDE
ggauggugaggcugugggu

SEQ ID NO: 27 - RNA, 19 nt, Artificial Sequence
  A SYNTHETIC SMALL INTERFERING NUCLEOTIDE
acucgacggccuccuguau

SEQ ID NO: 28 - RNA, 19 nt, Artificial Sequence
  A SYNTHETIC SMALL INTERFERING NUCLEOTIDE
ugaagaaccgagcggccag

SEQ ID NO: 29 - RNA, 19 nt, Artificial Sequence
  A SYNTHETIC SMALL INTERFERING NUCLEOTIDE
gucuuggauucgugaacag

SEQ ID NO: 30 - RNA, 19 nt, Artificial Sequence
  A SYNTHETIC SMALL INTERFERING NUCLEOTIDE
cguaguuugcgccuggcca

SEQ ID NO: 31 - RNA, 19 nt, Artificial Sequence
  A SYNTHETIC SMALL INTERFERING NUCLEOTIDE
gcaggaugcugugacucga

SEQ ID NO: 32 - RNA, 19 nt, Artificial Sequence
  A SYNTHETIC SMALL INTERFERING NUCLEOTIDE
aguaucaaccacgugucaa

SEQ ID NO: 33 - RNA, 19 nt, Artificial Sequence
  A SYNTHETIC SMALL INTERFERING NUCLEOTIDE
gaagaaccgagcggccaga

SEQ ID NO: 34 - RNA, 19 nt, Artificial Sequence
  A SYNTHETIC SMALL INTERFERING NUCLEOTIDE
ugcugugacucgacggccu

SEQ ID NO: 35 - RNA, 19 nt, Artificial Sequence
  A SYNTHETIC SMALL INTERFERING NUCLEOTIDE
ucgacggccuccuguauug

SEQ ID NO: 36 - RNA, 19 nt, Artificial Sequence
  A SYNTHETIC SMALL INTERFERING NUCLEOTIDE
ggccuccuguauuggacga

SEQ ID NO: 37 - RNA, 19 nt, Artificial Sequence
  A SYNTHETIC SMALL INTERFERING NUCLEOTIDE
aagcacaaacaaaucagcu

SEQ ID NO: 38 - RNA, 19 nt, Artificial Sequence
  A SYNTHETIC SMALL INTERFERING NUCLEOTIDE
guauuggacgagucuugga

SEQ ID NO: 39 - RNA, 19 nt, Artificial Sequence
  A SYNTHETIC SMALL INTERFERING NUCLEOTIDE
agaaccgagcggccagaac

SEQ ID NO: 40 - RNA, 19 nt, Artificial Sequence
  A SYNTHETIC SMALL INTERFERING NUCLEOTIDE
gcagcgcugucucccugaa

SEQ ID NO: 41 - RNA, 19 nt, Artificial Sequence
  A SYNTHETIC SMALL INTERFERING NUCLEOTIDE
gucacguccucuggcguca

SEQ ID NO: 42 - RNA, 19 nt, Artificial Sequence
  A SYNTHETIC SMALL INTERFERING NUCLEOTIDE
cgagucuuggauucgugaa

SEQ ID NO: 43 - RNA, 19 nt, Artificial Sequence
  A SYNTHETIC SMALL INTERFERING NUCLEOTIDE
ccuccuguauuggacgagu

SEQ ID NO: 44 - RNA, 19 nt, Artificial Sequence
  A SYNTHETIC SMALL INTERFERING NUCLEOTIDE
auuggacgagucuuggauu

SEQ ID NO: 45 - RNA, 19 nt, Artificial Sequence
  A SYNTHETIC SMALL INTERFERING NUCLEOTIDE
uuggauucgugaacagacc

SEQ ID NO: 46 - RNA, 19 nt, Artificial Sequence
  A SYNTHETIC SMALL INTERFERING NUCLEOTIDE
ggauucgugaacagaccac

SEQ ID NO: 47 - RNA, 19 nt, Artificial Sequence
  A SYNTHETIC SMALL INTERFERING NUCLEOTIDE
gugucaaggcaggucacgu

SEQ ID NO: 48 - DNA, 29 bp, Artificial Sequence
  A SYNTHETIC PRIMER
ggcaagtggatctcttgtgaattcttgac

SEQ ID NO: 49 - DNA, 27 bp, Artificial Sequence
  A SYNTHETIC PRIMER
gtagcacccaccttggtgagctggtac

SEQ ID NO: 50 - DNA, 30 bp, Artificial Sequence
  A SYNTHETIC FORWARD PRIMER
agttcgtgcggctgcgcaacaagtccaacg

SEQ ID NO: 51 - DNA, 28 bp, Artificial Sequence
  A SYNTHETIC REVERSE PRIMER
gtcatcaaaggatcgtcaccattctgac

SEQ ID NO: 52 - DNA, 27 bp, Artificial Sequence
  A SYNTHETIC REVERSE PRIMER
ccattcgaccaccaagcgaaacatcgc

SEQ ID NO: 53 - DNA, 21 bp, Artificial Sequence
  A SYNTHETIC PRIMER P177
gggacagccaggctattgatt

SEQ ID NO: 54 - DNA, 19 bp, Artificial Sequence
  A SYNTHETIC PRIMER P178
catggcttgtgctcgagga

SEQ ID NO: 55 - DNA, 20 bp, Artificial Sequence
  A SYNTHETIC PRIMER P1213
cttcttaccaggtgccttcg

SEQ ID NO: 56 - DNA, 20 bp, Artificial Sequence
  A SYNTHETIC PRIMER P1379
gaatcgtccaccctctgtgt

SEQ ID NO: 57 - DNA, 20 bp, Artificial Sequence
  A SYNTHETIC PRIMER P140
tattgtgtctgccgtgaatc

SEQ ID NO: 58 - DNA, 21 bp, Artificial Sequence
  A SYNTHETIC PRIMER P141
gccgtcttggtctcataggtc

SEQ ID NO: 59 - DNA, 19 bp, Artificial Sequence
  A SYNTHETIC PRIMER GAPDH-F
tcaccaccatggagaaggc

SEQ ID NO: 60 - DNA, 20 bp, Artificial Sequence
  A SYNTHETIC PRIMER GAPDH-R
gctaagcagttggtggtgca

SEQ ID NO: 61 - DNA, 27 bp, Artificial Sequence
  A SYNTHETIC PRIMER hsSUN1-F
ggacgtgtttaaacccacgacttctcg

SEQ ID NO: 62 - DNA, 28 bp, Artificial Sequence
  A SYNTHETIC PRIMER hsSUN1-R
ctctgactttagctgatccagctccagc

SEQ ID NO: 63 - DNA, 20 bp, Artificial Sequence
  A SYNTHETIC PRIMER GAPDH-F
agccacatcgctcagacacc

SEQ ID NO: 64 - DNA, 20 bp, Artificial Sequence
  A SYNTHETIC PRIMER GAPDH-R
gtactcagcggccagcatcg

SEQ ID NO: 65 - RNA, 25 nt, Artificial Sequence
  A SYNTHETIC STEALTH SIRNA DUPLEX
ccauccugaguauaccugucuguau

(19) United States
(12) Patent Application Publication
(10) Pub. No.: US 2015/0211004 A1
(43) Pub. Date: Jul. 30, 2015

(54) RNAI-BASED THERAPIES FOR CARDIOMYOPATHIES, MUSCULAR DYSTROPHIES AND LAMINOPATHIES

(71) Applicant: Agency for Science, Technology and Research, Singapore (SG)

(72) Inventors: Colin Stewart, Singapore (SG); Brian Burke, Singapore (SG)

(21) Appl. No.: 14/395,691

(22) PCT Filed: Apr. 22, 2013
(86) PCT No.: PCT/SG2013/000158

§ 371 Date: Oct. 20, 2014

Related U.S. Application Data
(60) Provisional application No. 61/687,222, filed on Apr. 20, 2012.

(51) Int. Cl. C12N 15/113; C12Q 1/68; G01N 33/68

(52) U.S. Cl. CPC C12N 15/113; G01N 33/6875; C12Q 1/6883; C12N 2310/14; G01N 2333/47; G01N 2800/52; C12Q 2600/158; C12Q 2600/156

(57) ABSTRACT

The present disclosure relates to inhibitor of Sun1 for treatment of laminopathies and to Sun1 as markers indicative of a patient's responsiveness to treatment, enabling improved prediction of a patient's risk, monitoring of laminopathies.

### CROSS-REFERENCE TO RELATED APPLICATIONS

This application claims the benefit of priority of U.S. provisional application No. 61/687,222, filed Apr. 20, 2012, the contents of it being hereby incorporated by reference in its entirety for all purposes.

### TECHNICAL FIELD

The present invention generally relates to the field of biochemistry and medicine. In particular, the present invention refers to the identification of Sun 1 inhibitors that are useful in treating laminopathies.

### BACKGROUND

The nuclear lamina, that underlies the inner nuclear membrane (INM), is a meshwork of type V intermediate filament proteins consisting primarily of the A and B type lamins. Mammalian somatic cells express four major types of lamins, including A and C encoded by the Lmna gene, and B1 and B2, each encoded by their own genes (Lmnb1 and 2). In addition to providing mechanical strength to the nucleus, recent discoveries in nuclear-lamina associated human diseases have established intimate connections between the nuclear envelope/lamina, and processes such as gene expression, DNA repair, cell cycle progression and chromatin organization.

Some 28 diseases/anomalies (the nuclearenvelopathies) are linked to mutations within proteins of the nuclear envelope and lamina, with about half the diseases arising from mutations in the Lamin genes, predominately LMNA. These disease phenotypes range from cardiac and skeletal myopathies, lipodystrophies, peripheral neuropathies, to premature aging with early death.

Two notable laminopathies are the autosomal dominant form of Emery-Dreifuss Muscular Dystrophy (AD-EDMD) that results in muscle wasting and cardiomyopathy and Hutchinson-Gilford progeria syndrome (HGPS), a rare genetic premature aging disease, where affected individuals expire with a mean life span of 13 years.

AD-EDMD is caused by missense mutations and/or deletions throughout the LMNA gene that generally disrupt the integrity of the lamina, resulting in mechanical weakening of the nucleus, making it more vulnerable to mechanically induced stress.

With HGPS, most cases arise from a single heterozygous mutation at codon 1824 of LMNA. This mutation produces an in-frame deletion of 50 amino acids, generating a truncated form of LAΔ50 lamin A, termed progerin, which remains farnesylated. HGPS individuals are overtly normal at birth with the disease manifesting around 18 months. The current view is that the permanently farnesylated progerin is affixed to the nuclear membrane, resulting in a toxic gain of function that elicits HGPS. How farnesylated progerin triggers HGPS is not understood.

If patients suffering from symptoms of laminopathies can be diagnosed early, pacemaker implantation can be lifesaving. There is currently no cure for laminopathies, including EDMD. Symptoms of the disease may be treated by, for example, physical therapy, corrective orthopedic surgery, pacemaker installation, and pharmaceutical intervention to, e.g., control seizures and the effects of lipodystrophy.

There is a need to provide improved treatment for laminopathies, and in particular congenital dilated cardiomyopathy and the autosomal dominant form of Emery-Dreifuss Muscular Dystrophy (AD-EDMD) that overcomes, or at least ameliorates, one or more of the disadvantages described above.

There is also a need to provide methods of treatment, of diagnosing and of monitoring laminopathies.

### SUMMARY

In a first aspect, there is provided a Sun1 inhibitor for use in treating a laminopathy.

In a second aspect, there is provided the use of a Sun1 inhibitor as described herein in the manufacture of a medicament for treating a laminopathy.

In a third aspect, there is provided a method of treating a laminopathy comprising the administration of an effective amount of a Sun1 inhibitor as described herein to a mammal in need thereof.

In a fourth aspect, there is provided an siRNA having a sequence which is complementary to the Sun1 mRNA sequence.

In a fifth aspect, there is provided an oligonucleotide having a sequence according to any one of SEQ ID NOs: 1 to 47.

In a sixth aspect, there is provided a method of diagnosing a laminopathy, or determining if an individual is at risk of developing a laminopathy, comprising the steps of:

(a) measuring the expression level of Sun1 in an individual or a sample obtained from the individual;
(b) comparing the Sun1 expression levels obtained from step (a) with a control reference wherein an elevated level of Sun1 in the individual compared to the control indicates that the individual has a laminopathy or is at risk of developing a laminopathy.

In a seventh aspect, there is provided a method of monitoring the progression or treatment of a laminopathy, comprising the steps of:

(a) measuring the expression level of Sun1 in an individual or a sample obtained from the individual;
(b) comparing the Sun1 expression levels obtained from step (a) with a control reference wherein an elevated level of Sun1 in the individual compared to the control indicates that the laminopathy has progressed from a less advanced stage to a more advanced stage.

### DEFINITIONS

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

Units, prefixes, and symbols are denoted in their Systeme International d'Unités (SI) accepted form. Numeric ranges are inclusive of the numbers defining the range. Unless otherwise indicated, nucleic acids are written left to right in 5′ to 3′ orientation. The headings provided herein are not limitations of the various aspects or embodiments of the invention, which can be had by reference to the specification as a whole. Accordingly, the terms defined immediately below are more fully defined by reference to the specification in its entirety.

The following words and terms used herein shall have the meaning indicated:

Analog, derivative or mimetic: An analog is a molecule that differs in chemical structure from a parent or reference compound, for example a homolog (differing by an incremental change in the chemical structure, such as a difference in the length of an alkyl chain), a molecular fragment, a structure that differs by one or more functional groups, a change in ionization. Structural analogs are often found using quantitative structure activity relationships (QSAR), with techniques known in the art. A derivative is a substance related to a base structure, and theoretically derivable from the base structure. A mimetic is a biomolecule that mimics the activity of another biologically active molecule. Biologically active molecules can include chemical structures that mimic the biological activities of a compound, for instance a native siRNA.

As used herein, the term “antisense strand” is meant to refer to a polynucleotide or region of a polynucleotide that is at least substantially (e.g., about 80% or more) or 100% complementary to a target nucleic acid of interest. Also, the antisense strand of a dsRNA is at least substantially complementary to its sense strand. An antisense strand may be comprised of a polynucleotide region that is RNA, DNA, or chimeric RNA/DNA. Additionally, any nucleotide within an antisense strand can be modified by including substituents coupled thereto, such as in a 2′ modification. The antisense strand can be modified with a diverse group of small molecules and/or conjugates. For example, an antisense strand may be complementary, in whole or in part, to a molecule of messenger RNA (“mRNA”), an RNA sequence that is not mRNA including non-coding RNA (e.g., tRNA and rRNA), or a sequence of DNA that is either coding or non-coding. The terms “antisense strand” and “antisense region” are intended to be equivalent and are used interchangeably.

The antisense region or antisense strand may be part of a larger strand that comprises nucleotides other than antisense nucleotides. For example, in the case of a unimolecular structure the larger strand would contain an antisense region, a sense region and a loop region, and might also contain overhang nucleotides and additional stem nucleotides that are complementary to other stem nucleotides, but not complementary to the target. In the case of a fractured hairpin, the antisense region may be part of a strand that also comprises overhang nucleotides and/or a loop region and two other regions that are self-complementary.

As used herein, the term “2′ carbon modification” refers to a nucleotide unit having a sugar moiety, for example a moiety that is modified at the 2′ position of the sugar subunit. A “2′-O-alkyl modified nucleotide” is modified at this position such that an oxygen atom is attached both to the carbon atom located at the 2′ position of the sugar and to an alkyl group. Examples include 2′-O-methyl, 2′-O-ethyl, 2′-O-propyl, 2′-O-isopropyl, 2′-O-butyl, 2-O-isobutyl, 2′-O-ethyl-O-methyl (—OCH₂CH₂OOCH₃), 2′-O-ethyl-OH (—OCH₂CH₂OH) and the like. A “2′ carbon sense modification” refers to a modification at the 2′ carbon position of a nucleotide on the sense strand or within a sense region of polynucleotide. A “2′ carbon antisense modification” refers to a modification at the 2′ carbon position of a nucleotide on the antisense strand or within an antisense region of polynucleotide.

As described herein, the phrase “gene silencing” or the word “silencing” refers to a process by which the expression of a specific gene product is lessened or attenuated. Gene silencing can take place by a variety of pathways. Unless specified otherwise, as used herein, gene silencing refers to decreases in gene product expression that results from Ribonucleic acid interference (RNAi), a defined, though partially characterized pathway whereby small inhibitory RNA (siRNA) act in concert with host proteins (e.g., the RNA induced silencing complex, RISC) to degrade messenger RNA (mRNA) in a sequence-dependent fashion. The level of gene silencing can be measured by a variety of means, including, but not limited to, measurement of transcript levels by Northern Blot Analysis, B-DNA techniques, transcription-sensitive reporter constructs, expression profiling (e.g., DNA chips), and related technologies. Alternatively, the level of silencing can be measured by assessing the level of the protein encoded by a specific gene. This can be accomplished by performing a number of studies including Western Analysis, measuring the levels of expression of a reporter protein that has e.g., fluorescent properties (e.g., GFP) or enzymatic activity (e.g., alkaline phosphatases), or several other procedures.

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated.

Perfect complementarity or 100% complementarity refers to the situation in which each nucleotide unit of one polynucleotide strand can hydrogen bond with a nucleotide unit of a second polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity.

The term “deoxynucleotide” refers to a nucleotide or polynucleotide lacking a hydroxyl group (OH group) at the 2′ and/or 3′ position of a sugar moiety. Instead, it has a hydrogen bonded to the 2′ and/or 3′ carbon. Within an RNA molecule that comprises one or more deoxynucleotides, “deoxynucleotide” refers to the lack of an OH group at the 2′ position of the sugar moiety, having instead a hydrogen, bonded directly to the 2′ carbon.

The terms “deoxyribonucleotide” and “DNA” refer to a nucleotide or polynucleotide comprising at least one sugar moiety that has an H, rather than an OH, at its 2′ and/or 3′position.

The phrase “duplex region” refers to the region in two complementary or substantially complementary polynucleotides that form base pairs with one another, either by Watson-Crick base pairing or any other manner that allows for a stabilized duplex between polynucleotide strands that are complementary or substantially complementary. For example, a polynucleotide strand having 21 nucleotide units can base pair with another polynucleotide of 21 nucleotide units, yet only 19 bases on each strand are complementary or substantially complementary, such that the “duplex region” has 19 base pairs. The remaining bases may, for example, exist as 5′ and 3′ overhangs. Further, within the duplex region, 100% complementarity is not required; substantial complementarity is allowable within a duplex region. Substantial complementarity refers to 79% or greater complementarity. For example, a mismatch in a duplex region consisting of 19 base pairs results in 94.7% complementarity, rendering the duplex region substantially complementary.

As used herein “inhibiting” or “treating” a disease refers to the following. Inhibiting the full development of a disease, disorder or condition, for example, in a subject who is at risk for a disease such as a laminopathy, an aging-associated disease or condition, atherosclerosis or cardiovascular disease. “Treatment” refers to a therapeutic intervention that ameliorates a sign or symptom of a disease or pathological condition after it has begun to develop. As used herein, the term “ameliorating,” with reference to a disease, pathological condition or symptom, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease or condition.

As used herein the term “isolated” biological component (such as a nucleic acid molecule, protein or organelle) has been substantially separated or purified away from other biological components in the cell of the organism in which the component naturally occurs, e.g., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

The term “miRNA” refers to microRNA. MicroRNAs (miRNAs) are single-stranded noncoding RNAs of 21-23 nucleotides. As used herein, the term miRNA mimic refers to a single-stranded RNA, chemically synthetized or isolated, capable of reproducing the function, structure and activity of a naturally occurring miRNA.

As used herein the term “morpholino oligomer” refers to a polymeric molecule having a backbone which supports bases capable of hydrogen bonding to typical polynucleotides, wherein the polymer lacks a pentose sugar backbone moiety, and more specifically a ribose backbone linked by phosphodiester bonds which is typical of nucleotides and nucleosides, but instead contains a ring nitrogen with coupling through the ring nitrogen. A morpholino oligomer is composed of “morpholino subunit” structures, such as shown below, which in the oligomer are preferably linked together by phosphoramidate or phosphorodiamidate linkages, or their thio analogs, joining the morpholino nitrogen of one subunit to the 5′ exocyclic carbon of an adjacent subunit. Each subunit includes a purine or pyrimidine base-pairing moiety Pi which is effective to bind, by base-specific hydrogen bonding, to a base in a polynucleotide.

The term “phosphorodiamidate” group as used herein comprises phosphorus having two attached oxygen atoms and two attached nitrogen atoms, and herein may also refer to phosphorus having one attached oxygen atom and three attached nitrogen atoms. In the intersubunit linkages of the oligomers described herein, one nitrogen is typically pendant to the backbone chain, and the second nitrogen is the ring nitrogen in a morpholino ring structure. Alternatively or in addition, a nitrogen may be present at the 5′-exocyclic carbon.

The term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs.

Nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

Modified bases refer to nucleotide bases such as, for example, adenine, guanine, cytosine, thymine, uracil, xanthine, inosine, and queuosine that have been modified by the replacement or addition of one or more atoms or groups. Some examples of types of modifications that can comprise nucleotides that are modified with respect to the base moieties include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any 0- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

The term nucleotide is also meant to include what are known in the art as universal bases. By way of example, universal bases include but are not limited to 3-nitropyrrole, 5-nitroindole, or nebularine. The term “nucleotide” is also meant to include the N3′ to P5′ phosphoramidate, resulting from the substitution of a ribosyl 3′ oxygen with an amine group.

Further, the term nucleotide also includes those species that have a detectable label, such as for example a radioactive or fluorescent moiety, or mass label attached to the nucleotide.

As used herein, the term “nucleic acid” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA” is a DNA that has undergone a molecular biological manipulation.

The phrases “off-target silencing” and “off-target interference” are defined as degradation of mRNA other than the intended target mRNA due to overlapping and/or partial homology with secondary mRNA messages.

As used herein, the term “oligonucleotide” refers to a short, single-stranded nucleic acid molecule. An oligonucleotide is a plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Particular oligonucleotides and oligonucleotide analogs can include linear sequences up to about 200 nucleotides in length, for example a sequence (such as DNA or RNA) that is at least 6 bases, for example at least 8, 10, 15, 20, 25, 30, 35, 40, 45, 50, 100 or even 200 bases long, or from about 6 to about 50 bases, for example about 10-25 bases, such as 12, 15 or 20 bases.

Oligonucleotides composed of 2′-deoxyribonucleotides (oligodeoxyribonucleotides) are fragments of DNA and are often used in the polymerase chain reaction, a procedure that can greatly amplify almost any small amount of DNA. There, the oligonucleotide is referred to as a primer, allowing DNA polymerase to extend the oligonucleotide and replicate the complementary strand.

As used herein the term Peptide Nucleic Acid (PNA) refers to an oligonucleotide analog with a backbone comprised of monomers coupled by amide (peptide) bonds, such as amino acid monomers joined by peptide bonds.

The term “polynucleotide” refers to polymers of nucleotides, and includes but is not limited to DNA, RNA, DNA/RNA hybrids including polynucleotide chains of regularly and/or irregularly alternating deoxyribosyl moieties and ribosyl moieties (i.e., wherein alternate nucleotide units have an —OH, then and then an —OH, then an —H, and so on at the 2′ position of a sugar moiety), and modifications of these kinds of polynucleotides, wherein the attachment of various entities or moieties to the nucleotide units at any position are included.

The term “polyribonucleotide” refers to a polynucleotide comprising two or more modified or unmodified ribonucleotides and/or their analogs. The term “polyribonucleotide” is used interchangeably with the term “oligoribonucleotide.”

The term “ribonucleotide” and the phrase “ribonucleic acid” (RNA), refer to a modified or unmodified nucleotide or polynucleotide comprising at least one ribonucleotide unit. A ribonucleotide unit comprises an hydroxyl group attached to the 2′ position of a ribosyl moiety that has a nitrogenous base attached in N-glycosidic linkage at the 1′ position of a ribosyl moiety, and a moiety that either allows for linkage to another nucleotide or precludes linkage.

As used herein, the term “RNA interference” or “RNAi” are synonymous and refer to the process by which a polynucleotide, siRNA, shRNA or fractured shRNA comprising at least one ribonucleotide unit exerts an effect on a biological process. The process includes, but is not limited to, gene silencing by degrading mRNA, attenuating translation, interactions with tRNA, rRNA, hnRNA, miRNA, cDNA and genomic DNA, as well as methylation of DNA, and/or methylation or acetylation of proteins (e.g., histones) associated with DNA. As used herein, the term “sense strand” is meant to refer to a polynucleotide or region that has the same nucleotide sequence, in whole or in part, as a target nucleic acid such as a messenger RNA or a sequence of DNA. The term “sense strand” includes the sense region of a polynucleotide that forms a duplex with an antisense region of another polynucleotide.

Also, a sense strand can be a first polynucleotide sequence that forms a duplex with a second polynucleotide sequence on the same unimolecular polynucleotide that includes both the first and second polynucleotide sequences. As such, a sense strand can include one portion of a unimolecular siRNA that is capable of forming hairpin structure, such as an shRNA. When a sequence is provided, by convention, unless otherwise indicated, it is the sense strand or region, and the presence of the complementary antisense strand or region is implicit. The phrases “sense strand” and “sense region” are intended to be equivalent and are used interchangeably.

The sense region or sense strand may be part of a larger strand that comprises nucleotides other than sense nucleotides. For example, in the case of a unimolecular structure the larger strand would contain a sense region, an antisense region and a loop region, and might also contain overhang nucleotides and additional stem nucleotides that are complementary to other stem nucleotides, but not complementary to the target. In the case of a fractured hairpin, the sense region may be part of a strand that also comprises overhang nucleotides and/or a loop region and two other regions that are self-complementary.

As used herein, the term “siRNA” is meant to refer to a small inhibitory RNA duplex that induces gene silencing by operating within the RNA interference (“RNAi”) pathway. These molecules can vary in length (generally 18-30 base pairs) and contain varying degrees of complementarity to their target mRNA in the antisense strand. Some, but not all, siRNA have unpaired overhanging bases on the 5′ or 3′ end of the sense strand and/or the antisense strand. The term “siRNA” includes duplexes of two separate strands, as well as single strands that can form hairpin structures comprising a duplex region.

Each siRNA can include between 17 and 31 base pairs, more preferably between 18 and 26 base pairs, and most preferably 19 and 21 base pairs. Some, but not all, siRNA have unpaired overhanging nucleotides on the 5′ and/or 3′ end of the sense strand and/or the antisense strand. Additionally, the term “siRNA” includes duplexes of two separate strands, as well as single strands that can form hairpin structures comprising a duplex region, which may be referred to as short hairpin RNA (“shRNA”).

siRNA may be divided into five (5) groups (non-functional, semi-functional, functional, highly functional, and hyper-functional) based on the level or degree of silencing that they induce in cultured cell lines. As used herein, these definitions are based on a set of conditions where the siRNA is transfected into said cell line at a concentration of 100 nM and the level of silencing is tested at a time of roughly 24 hours after transfection, and not exceeding 72 hours after transfection. In this context, “non-functional siRNA” are defined as those siRNA that induce less than 50% (<50%) target silencing. “Semi-functional siRNA” induce 50-79% target silencing. “Functional siRNA” are molecules that induce 80-95% gene silencing. “Highly-functional siRNA” are molecules that induce greater than 95% gene silencing. “Hyperfunctional siRNA” are a special class of molecules. For purposes of this document, hyperfunctional siRNA are defined as those molecules that: (1) induce greater than 95% silencing of a specific target when they are transfected at subnanomolar concentrations (i.e., less than one nanomolar); and/or (2) induce functional (or better) levels of silencing for greater than 96 hours. These relative functionalities (though not intended to be absolutes) may be used to compare siRNAs to a particular target for applications such as functional genomics, target identification and therapeutics.

As used herein, the terms “shRNA” or “hairpins” are meant to refer to unimolecular siRNA comprised by a sense region coupled to an antisense region through a linker region. A shRNA may have a loop as long as, for example, 4 to 30 or more nucleotides. In some embodiments it may be preferable not to include any non-nucleotides moieties. The shRNA may also comprise RNAs with stem-loop structures that contain mismatches and/or bulges, micro-RNAs, and short temporal RNAs. RNAs that comprise any of the above structures can include structures where the loops comprise nucleotides, non-nucleotides, or combinations of nucleotides and non-nucleotides. The sense strand and antisense strand of an shRNA are part of one longer molecule or, in the case of fractured hairpins, two (or more) molecules that form a fractured hairpin structure.

The phrase “substantially similar” refers to a similarity of at least 90% with respect to the identity of the bases of the sequence.

The term “target” is used in a variety of different forms throughout this document and is defined by the context in which it is used. “Target mRNA” refers to a messenger RNA to which a given siRNA can be directed against. “Target sequence” and “target site” refer to a sequence within the mRNA to which the sense strand of a siRNA shows varying degrees of homology and the antisense strand exhibits varying degrees of complementarity. The phrase “siRNA target” can refer to the gene, mRNA, or protein against which a siRNA is directed. Similarly, “target silencing” can refer to the state of a gene, or the corresponding mRNA or protein.

By “therapeutic” or “treating” is meant the amelioration of the laminopathy, itself, and the protection, in whole or in part, against further progression of the laminopathy. By “prophylactic” or “preventing” or “inhibiting” is meant the protection, in whole or in part, against laminopathy, and symptoms associated therewith. “Preventing” also can entail slowing (or delaying) the onset of laminopathy in a subject. One of ordinary skill in the art will appreciate that any degree of protection from or amelioration of, a laminopathy or symptom associated therewith is beneficial to a subject, such as a human patient. For example, the inventive method may reduce the severity of symptoms in a subject and/or delay the appearance of symptoms, which improves the quality of life of the subject.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is a series of plots showing defects in body weight and longevity, in the Lmna^{-/-} and Lmna^{L530P/L530P} (LmnaΔ9 mice) mice are ameliorated in the homozygous Sun1 knockout Lmna^{-/-}Sun1^{-/-} and LmnaΔ9Sun1^{-/-} animals.

FIG. 1 A is a line chart showing body weights of mice with the indicated genotypes. Values are the averages from animals in each cohort. The number (n) of animals in each cohort used for weight measurements is indicated.

FIG. 1 B is a Kaplan-Meier graph showing significantly increased life span of Lmna^{-/-} Sun1^{-/-} mice compared to Lmna^{-/-} mice. The median survival of wild type or Sun1^{-/-} is >210 days during a 7 month follow up; Lmna^{-/-} mice have median survival of 41 days; Lmna^{-/-} Sun1^{+/-} mice have a median of 54 days; and Lmna^{-/-}Sun1^{-/-} mice have a median of 104 days (P<0.01 comparing Lmna^{-/-} and Lmna^{-/-}Sun1^{-/-}).

FIG. 1 C is a line chart showing body weights of LmnaΔ9 mice that are wild type, heterozygous, or homozygous for Sun1defficiency. The wild type and Sun1^{-/-} cohorts are graphed in parallel for comparison. Values are the averages from animals in each cohort. The number (n) of animals in each cohort is indicated. (P<0.0001 comparing LmnaΔ9Sun1^{+/+} and LmnaΔ9Sun1^{-/-}).

FIG. 1 D is a Kaplan-Meier graph showing the increased life span of LmnaΔ9Sun1^{-/-} compared to LmnaΔ9Sun1^{+/+} mice. LmnaΔ9Sun1^{+/-} mice are also graphed for comparison. (P<0.0001 comparing LmnaΔ9Sun1^{+/+} and LmnaΔ9Sun1^{-/-}).

FIG. 1 E is a line chart showing cell proliferation curves of Mouse Embryonic Fibroblasts (MEFs) with the indicated genotypes. The MEF growth curves are representative of >3 independent isolates from embryos of the indicated genotypes. Relevant P values are indicated in the graph.

FIG. 1 F is a line chart showing cell proliferation curves of MAFs (mouse adult fibroblasts) from WT, Sun1^{-/-}, LmnaΔ9Sun1^{+/+} and LmnaΔ9Sun1^{-/-} mice. MAFs were seeded on E-plates 96 (Roche) at a density of 1000 cells per well. Cell growth was measured with the xCELLigence system (Roche). Normalized cell indexes obtained from xCELLigence are presented. Relevant P values are indicated in the graph.

FIG. 2 relates to correction of the Lmna^{-/-} skeletal and multiple tissue defects in the Lmna^{-/-}Sun1^{-/-} double knock out mouse.

FIG. 2 A is a series micro-CT scans of the indicated mice. The Lmna^{-/-} mice display a lordokyphosis (curvature of the spinal column) phenotype corrected in Lmna^{-/-}Sun1^{-/-} mice.

FIG. 2 B is a series of three-dimensional images from micro-CT analyses and bar graphs. Three-dimensional images from micro-CT analyses of the femoral trabeculae from 40-day-old mice (left panels). Thinner trabecular formation was observed in the Lmna^{-/-} mouse compared to the other genotypes. The right panels quantify bone density (upper) and the number of trabeculaes/mm (lower) in the indicated genotypes. P values (right panels) of the differences between the indicated genotypes are shown.

FIG. 2 C is a series of hematoxylin and eosin (H&E)-stained cross sections of tissues from 5-6 week old mice. In each case, Lmna^{-/-}Sun1^{-/-} tissues are improved in pathology over Lmna^{-/-} counterparts. Cardiac muscle: Lmna^{-/-} cardiac muscle showed more tissue vacuoles than WT, Sun1^{-/-}, or Lmna^{-/-}Sun1^{-/-} muscle (600× magnifications). Hollow triangle: infiltrates of lymphocytes and neutrophils, solid triangle: sarcoplasmic vacuoles, arrow: myocyte necrosis.

FIG. 2 D is a bar graph showing cardiac function. Abnormal cardiovascular functions were found in the Lmna^{-/-} mice; the left ventricle ejection fraction as indicated in the “Cardiovascular Parameters” was measured by MRI (magnetic resonance imaging). Values are mean±SD.

FIG. 2 E is a series of hematoxylin and eosin (H&E)-stained cross sections of tricep muscle and quadricep femoris muscle from 5-6 week old mice. the musculature of Lmna^{-/-} mice contains smaller myocytes; the nuclei are closer together, and the myocytes adjacent to the bone are significantly atrophied (600× magnifications).

FIG. 3. Relates to extranuclear Sun1 accumulation in the Golgi of Lmna^{-/-} MEFs.

FIG. 3 A is a series of confocal microscopy images of the indicated cells. Cells were co-immunostained with anti-lamin A (green) and anti-Sun1 (red) antibodies. Extranuclear Golgi localization of Sun1 is seen in Lmna^{-/-} MEFs.

FIG. 3 B is a box plot showing quantification of Sun1 expression in MEFs. Mean±s.d. reflects collective results from two separate experiments with n=29 (WT) and n=36 (Lmna^{-/-}) MEFs. The difference between WT and Lmna^{-/-} is statistically significant (P<0.0001).

FIG. 3 C is series of confocal microscopy images of the indicated cells. WT, Lmna^{-/-} and Lmna^{-/-}Sun1^{-/-} MEFs were stained with anti-Lamin B1 (red) and DAPI (blue). Lamin B1 nuclear envelope staining is intact in WT and Lmna^{-/-}Sun1^{-/-} MEFs with the staining being irregular with herniations in Lmna^{-/-} nuclei. Arrows point to disruptions in the nuclear envelope. Bars: 10 μm.

FIG. 3 D is a bar graph showing quantification of the prevalence of cells with visible nuclear envelope disruptions. The values are averages from three independently isolated MEFs of the indicated genotype (each counted for 300 nuclei). The prevalence of nuclear disruptions between Lmna^{-/-} and Lmna^{-/-}Sun1^{-/-} MEFs is significantly different (P<0.0001).

FIG. 3 E is a bar graph and western blotting image. The upper graph demonstrates that over expression of Sun1 in the absence of lamin A exacerbates nuclear herniations. WT and Lmna^{-/-}Sun1^{-/-} MEFs were transfected with increasing amounts of a mouse Sun1 (mSun1) expression vector. The nuclei were stained and visualized 48 hours after transfection. Values are averages from three experiments (each sample was counted for 300 nuclei per experiment). The lower panels show analysis by Western blotting for the expression of transfected Sun1 of cells transfected in parallel; actin signals are shown as loading controls.

FIG. 3 F is a series of dot plot showing FACS analysis of the indicated cells. WT (top) or Lmna^{-/-}Sun1^{-/-} (bottom) MEFs were transfected with vector-alone (left) or increasing amounts of mSun1 expressing plasmid (right three panels), and the cells were analyzed 48 hours later by FACS for propidium iodide (PI; Y-axis) and annexin V (X-axis). The percentage of apoptotic cells (in the lower right quadrant of the scans) is indicated.

FIG. 4 demonstrates that the loss of Lamin A correlates with Sun1 accumulation in the nuclear envelop and the Golgi.

FIG. 4 A is a series of confocal images of the indicated cells. Lmna−/− MEFs or LmnaΔ9 MAFs (mouse adult fibroblasts) were stained with anti-Sun1 (red) and anti-GM130 (a Golgi marker; green; right middle panels) or anti-Calnexin (an ER marker; green; left middle panels). DAPI staining of DNA is in blue. Yellow in merged panels indicates Sun1 colocalization with GM130 in the Golgi, and absence of colocalization with Calnexin in the ER. Localization of Sun1 in the Golgi was observed in Lmna−/− and LmnaΔ9 cells. Images are summations of z-stacks.

FIG. 4 B is a series of western blot of Golgi preparation using cytosolic lysate (S) from Lmna−/− liver tissue was fractionated on a sucrose density gradient; the Golgi fractions (F1-F9) were examined together and compared to total loading cytosolic lysate (S) by immunoblotting using anti-mouse Sun1 and anti-Golgi marker GM130, respectively. The mouse Sun1 protein cofractionated with Golgi constituent protein GM130. Golgi preparation from WT liver tissue fractionated in the same way is shown as control at the bottom. Unlike Lmna−/− liver cytosol, minimal Sun1 signal was detected in the WT cytosolic lysate (S).

FIG. 4 C is a dot plot and linear regression graph showing the correlation of Sun1 staining in the nucleus and Golgi in WT and Lmna−/− MEFs. Linear regression indicates a positive correlation (slope=0.375) between Sun1 in the nucleus and in the Golgi in Lmna−/− MEFs.

FIG. 4 D is a series of confocal immunofluorescent images showing localization of cell endogenous Sun2, Nup153, Emerin and transfected human Nesprin1 (accession number NM_{—}133650, 982 aa) in WT and Lmna−/− MEFs. Nuclear envelope localization of Sun2 and Nup153 was not perturbed by Lmna depletion while some increased cytoplasmic distribution of Emerin and Nesprin1 was seen in Lmna−/− MEFs. No workable antibody that recognizes cell endogenous Nespirin1 was available; so the analysis was performed with FLAG-tagged transfected Nespirin1 stained with anti-FLAG.

FIG. 4 E is a series of western blotting of Sun1, Sun2, Nup153, lamin B1, Emerin and α-tubulin in MEFs (left) and mouse liver tissue (right). Wild-type, Lmna−/−, and Sun1−/− samples were compared. Mouse identification (ID) numbers indicate individual animals. Aside from Sun1, no consistent difference was noted between Lmna−/− and WT cells or liver tissues.

FIG. 4 F is a series of images by ethidium bromide staining showing the RT-PCR analysis of Sun1 mRNA (nucleotides 250-408, 1213-1379 and 2168-2353) from wild-type (lane 1) and four individual Lmna−/− (lanes 2-5) MEFs. Gapdh is shown as control.

FIG. 5 shows analyses of Sun1 protein turnover.

FIG. 5 A is a series of immunofluorescence images of wild-type MEFs and Lmna−/− MEFs treated without or with 10 mM of lactacystin (for 14 hr). Cells were fixed and co-immunostained with rabbit anti-mSun1 (green) and mouse anti-GM130 (red) antibodies. DNA is in blue. Increased Sun1 is seen in the nucleus with some protein found in extranuclear locale of WT MEFs (in 10%-15% of cells, indicated by arrowheads) after lactacystin treatment. In lactacystin treated WT MEFs, Sun1 accumulation was observed in the nuclear membrane with a circumferential pattern and in the nucleoplasm with a punctate pattern. In Lmna−/− MEFs, Sun1 accumulation in the nucleus and in the Golgi is increased after lactacystin treatment.

FIG. 5 B is a series of western blots of Sun1 in wild-type and Lmna−/− MEFs treated without or with 25 mg/ml cycloheximide (for 12 or 24 hr); α-tubulin was used as a normalization control. Relative amounts of Sun1 were calculated and shown in the numbers below the blot. The half-life of the Sun1 protein is approximately 12 hr in WT MEFs and is calculated to approximate >24 hr in Lmna−/− MEFs.

FIG. 6 relates to the over expression of Golgi-targeted Sun1 increased nuclear aberrations and cell death.

FIG. 6 A is a series of confocal immunofluorescence images of wild type MEFs. Wild type MEFs were transfected with a FLAG-tagged mouse Sun1 expression vector. Transfected cells were co-stained with mouse anti-FLAG (green), rabbit anti-GM130 (red), and goat anti-lamin B1 (grey scale). A representative image of modest nuclear blebs and ruffles seen in some transfected cells is shown. Bars, 10 μm.

FIG. 6 B is a series of confocal images of a Golgi-targeted mouse Sun1 (fused with Tgn38, HA-tagged). A Golgi-targeted mouse Sun1 expression plasmid was transfected into WT MEFs. Thirty hours after transfection, cells were immunostained with mouse anti-HA (green), rabbit anti-GM130 (red), and goat anti-lamin B1 (grey scale). Distinct aberrancies are visualized by cytoplasmic lamin B1 staining (see arrow heads) of pmSun1-Tgn38-HA transfected cells. Bars, 10 μm.

FIG. 6 C is bar graph showing statistical quantification of the cytoplasmic release of lamin B1 in MEFs transfected (for 30 hours) with either mSun1 (mSun1-FLAG) or the Golgi-targeted mSun1 (pmSun1-Tgn38-HA). One hundred cells were counted in each case.

FIG. 7 relates to a human SUN1 deleted for its N-terminal lamin A-interacting domain showing that it locates in the Golgi.

FIG. 7 A is a series of confocal images showing the localization of WT or N-terminal deletion (amino acids 103-785) mutant of HA-tagged human SUN1 in MEFs. Cells were co-immunostained with mouse anti-HA (green), rabbit anti-GM130 (red) and goat anti-lamin B1 (gray scale). DNA was stained with Hoechst33342 (blue). SUN1 (103-785) mutant protein localizes to extranuclear Golgi; while WT SUN1 is in the nuclear membrane. The arrowheads denote cytoplasmic lamin B1. The scale bars represent 10 μm.

FIG. 7 B is a bar graph relating to the quantification of MEF cells with cytoplasmic release of lamin B1 in MEFs after 30 hr of transfection of HA-tagged wild-type human SUN1 or the SUN1 (103-785) mutant protein. One hundred cells were counted in each case.

FIG. 8 A to C are a series of immunofluorescent images and a bar graph showing the effect of brefeldin A, nocodazole and latrunculin on the indicated cells. FIG. 8 relates to the reduction of nuclear irregularities in Lmna^{-/-} MEFs in the presence of brefeldin A and nocodazole, but not latrunculin.

FIG. 8 A shows on the left, immunostaining of Sun1 (red) and GM130 (green) in Lmna^{-/-} MEFs treated for 24 hours with brefeldin A (BFA, 10 μg/mL). Note in treated cells the reduction of Sun1 and GM130 from the Golgi. (Right) Quantification of BFA treatment on the nuclear morphology of Lmna^{-/-} MEFs. Untreated and treated cells were stained with a mouse Sun1-specific antibody or with DAPI in cells passaged 4 (P4), and 8 (P8) times, respectively. The nuclear morphology was evaluated by observers blinded for genotype and by computerized image analyses of nuclear contours.

FIG. 8 B shows (Left) the sub-cellular localization of Sun1 in Lmna^{-/-} MEFs untreated or treated with 5 μM nocodazole for 4 hours. The Golgi complex was stained with a mouse antibody against GM130 (green) and a rabbit antibody against mouse Sun1 (red). (Middle) Parallel cells untreated and treated with nocodazole and stained for α-tubulin are shown. (Right) Quantification of nocodazole treatment on the nuclear morphology of Lmna−/− MEFs. The difference between untreated and treated cells is statistically significant (P=0.0058).

FIG. 8 C shows (Left) Lmna^{-/-} MEFs that were untreated or treated with 40 nM of latrunculin (LAT-B) for 12 hours. Cells were fixed and immunostained for Sun1 and GM130. (Middle) Parallel cells untreated and treated with latrunculin and visualized with fluorescent phalloidin for actin are shown. (Right) Quantification of LAT-B treatment on the nuclear morphology of Lmna^{-/-} MEFs. The difference between untreated and treated cells was statistically insignificant (P=0.8376).

FIG. 9 relates to the correlation between nuclear irregularities in HGPS fibroblasts and SUN1 expression.

FIG. 9 A is a series of confocal images showing immunostaining of SUN1 and lamin B1 in normal (AG03512 and AG03258) and HGPS (AG06297 and AG11498) skin fibroblasts. Cells were stained with anti-human SUN1 (green) and anti-lamin B1 (red) antibodies. DAPI staining is in blue. Yellow arrow heads point to cells expressing high-SUN1, white arrow heads to cells with low-SUN1.

FIG. 9 B is a series of confocal images showing the visualization of the nuclear morphologies of control (AG03512) and HGPS (AG11498) skin fibroblasts transfected using Lipofectamine 2000 with control-siRNA or SUN1-siRNA for 72 hours.

FIG. 9 C is a series of bar graphs showing the quantification of the integrated immunofluorescent intensities of SUN1 in cells treated with control or SUN1 siRNA from (B). One hundred twenty to two hundred cells from each of the indicated samples from (B) were visualized and quantified for staining intensities. The intensities were normalized to the average intensity of SUN1 in AG03512 cells. The cells with SUN1 staining intensities less than 2 fold different from average are represented by blue bar; the cells that are >2 fold, but <5 fold are represented by pink bar; the cells that stained >5 fold above average are represented by brown bar. *, P<0.001 when compared to AG03512 cells (t-test).

FIG. 9 D is a series of bar graph relating to the quantification of the prevalence of cells from (B) with nuclear irregularities. ‡, P<0.0001, when comparing the same cells treated with either control RNAi or SUN1-RNAi (Fisher's exact test).

FIG. 9 E is a bar graph relating to the quantification of aberrant nuclear morphology in normal and HGPS fibroblasts transfected with a human SUN1 expression plasmid tagged with HA. Two hundred mock transfected cells per sample and fifty transfected cells per sample were scored. P values were calculated by Fisher's exact test.

FIG. 10 shows the properties of normal human and Hutchinson-Gilford progeria syndrome skin fibroblasts.

FIG. 10 A is a series of confocal images showing immunofluorescent SUN1 staining images of multiple cells from four normal (AG03512, AG03257, AG03258, AG08469) and seven HGPS (AG01972, AG11513, AG06297, AG11498, AG06917, AG11513, AG03198) fibroblasts. Note that the increased expression of SUN1 is seen in all HGPS samples with one third or more of cells in each HGPS visual field staining brightly green. The scale bars represent 50 μm.

FIG. 10 B is a series of western blots showing the expression of SUN1, lamin A/C and progerin in normal and representative HGPS skin fibroblasts was assessed by western blotting. Progerin which is deleted for 50 amino acids from full length lamin A runs slightly faster in SDS-PAGE. Relative intensities of SUN1 expression levels compared to AG03512 (lanes 2-4) or AG08469 (lanes 6-8) are indicated in the numbers below the top panel.

FIG. 10 C is a series of ethidium bromide stained agarose gels showing the expression of SUN1 mRNA in normal and representative HGPS skin fibroblasts by RT-PCR. GAPDH was used for normalization.

FIG. 10 D is a cartoon of nuclei with shapes and contour changes that are scored as nuclear invaginations. Nuclei with >240° contour changes are scored as aberrant invagination(s).

FIG. 10 E is a pair of plots showing the distribution of nuclear invaginations in normal and HGPS skin fibroblasts (as presented in FIGS. 10B-10D) treated with control (siC) or SUN1 siRNA (siSUN1). Significantly higher numbers of aberrant nuclear invaginations (p<0.001) were seen for each of the HGPS cells compared to control AG03512 (normal skin fibroblasts, t test); similarly, significantly lower numbers of nuclear invaginations (p<0.0001) were seen for all HGPS cells treated with SUN1-RNAi compared to Control-RNAi treated cells (t test). RFU are relative fluorescent units of staining for SUN1.

FIG. 11 relates to the alleviation of HGPS-associated loss of NURD complex and cellular senescence after knock down of SUN1.

FIG. 11 A is a series of confocal images showing normal (AG03512) and HGPS (AG11498) skin fibroblasts were stained for heterochromatin markers (RBBP4 or H3K9me3; green) and SUN1 (red). Yellow arrow heads point to cells expressing high-SUN1; white arrow heads denote cells with low-SUN1.

FIG. 11 B is a scatter plot showing the expression levels of heterochromatin markers (RBBP4 or H3K9me3) and SUN1 in two normal and three HGPS skin fibroblasts were quantified by MetaMorph software. Each dot represents fluorescence intensity (in Log₁₀ scale) in a single cell of RBBP4 (left) or H3K9me3 (right) vs. SUN1. Linear curve fitting and correlation coefficient (r) for each plot are indicated. In HGPS cells, the expression of RBBP4 and H3K9me3 correlates negatively with the expression of SUN1.

FIG. 11 C is a series of confocal images and a dot plot showing the quantification of fluorescence from the images. HGPS fibroblasts (AG03513) were treated with control or SUN1 siRNA (for 72 hours by Lipofectamine RNAiMAX), and cells were stained with antibodies for SUN1 (red) and RBBP4 (green). Increased RBBP4 expression was observed in SUN1 siRNA-treated cells compared to control siRNA-treated cells. Graphic quantification of the staining intensities of RBBP4 vs. SUN1 in individual HGPS fibroblasts treated with control (blue) or SUN1 (brown) siRNA is shown (right); each dot represents a single cell (154 control and 157 SUN1 RNAi treated cells were quantified).

FIG. 11 D is a series of microscopic images and bar graph showing on the left panel the visualization of acidic senescence associated β-galactosidase (SA-β-Gal) in normal (AG03257) and HGPS (AG11498 at passage 8) fibroblasts transfected with Control- or SUN1-RNAi using Lipofectamine 2000 for 96 hours. On the right panel, a bar graph shows the quantification of the stained senescent cells. Standard deviations are from three independent assays counting between 1200 to 2000 cells in each experiment. Cell scoring was performed in a blinded fashion by an independent investigator. The P value (Chi-square) is indicated above the bars.

FIG. 11 E is a pair of scatter plot showing cell proliferation in normal and HGPS cells transfected with control or SUN1 RNAi. Cells at ˜50% of confluency were transfected with the siRNAs. When the cells reached confluency, equal numbers were seeded into dishes and quantified for proliferation. Cells were quantified 24 hours after cell seeding (day 0), and after another 4, 8, 10, 12 days of culturing using Cell Counting Kit-8. Relative absorbance at 460 nm was obtained by [(Absorbance₄₆₀ₙₘ-background Absorbance₄₆₀ₙₘ) at day N]/[(Absorbance₄₆₀ₙₘ-background Absorbance₄₆₀ₙₘ) at day 0]. Standard deviations were from triplicate experiments.

FIG. 12 relates to RBBP4 in the indicated cells. Lmna^{-/-}Sun1^{-/-} and WT mouse liver tissue show more RBBP4 staining than Lmna^{-/-} liver tissue.

FIG. 12 A is a series of confocal images of the indicated cells. WT and Lmna^{-/-} MEFs were stained with rabbit anti-RBBP4 (green). Note the reduced staining for RBBP4 in Lmna^{-/-} MEFs. DAPI staining of DNA is in blue.

FIG. 12 B is a series of microscopic images of liver tissue section as indicated below. Liver tissue from WT, Lmna^{-/-}, Sun1^{-/-} and Lmna^{-/-} Sun1^{-/-} was stained with RBBP4 by immunohistochemistry. Brown signals show nuclear RBBP4 staining; note fewer numbers of “brown” nuclei in Lmna^{-/-} liver compared to WT, Sun1^{-/-} and Lmna^{-/-} Sun1^{-/-} liver. Images are at 400× magnification.

FIG. 13 is a table and an immunofluorescence image relating to the efficacy of human Sun 1-specific siRNA.

FIG. 13 A is a table summarizing the efficacy of Sun 1 depletion at 48 h post transfection. The indicated siRNA were transfected in HeLa Cells and 48 hours post transfection the cells were fixed and Sun 1 protein was detected with a Sun 1 specific antibody. The number of cells expressing Sun 1 was assessed and compared with cells that were transfected with siRNA unrelated to Sun 1. The result were compared and summarized in the table. (++; +++; ++++) The (+) signs are subjective scores based upon immunofluorescence microscopy. (++++) indicates that cells contain no detectable Sun1 (i.e. the oligonucleotide is very effective). Untreated cells would have no (+) signs indicating that they had normal Sun1 levels. (++) and (+++) indicate partial levels of Sun1 depletion (i.e. that the oligonucleotides are partially, but not completely, effective under the transfection conditions chosen.

FIG. 13 B is an immunofluorescence microscopy image of HeLa cells treated for 48 h with the J-025277-08 UNC84A (SUN1) siRNA. Cells were labeled with an antibody against Sun1 (Red). Nuclei (Blue) are revealed with DAPI, a DNA-specific stain. This image reveals that at least 90% of the cells are depleted of Sun1.

### DETAILED DESCRIPTION

Before the present compounds and methods are described, it is to be understood that this invention is not limited to particular compounds, methods and experimental conditions described, as such compounds, methods, and conditions may vary. It is also to be understood that the terminology used herein is for purposes of describing particular embodiments only, and is not intended to be limiting, since the scope of the present invention will be limited only by the appended claims.

The invention is predicated, at least in part, on the surprising discovery that that Lmna^{-/-}, LmnaΔ9, and HGPS dysfunctions converge at a common pathogenic over accumulation of the inner nuclear envelope Sun1 protein in the Golgi. It was previously reported that the inner nuclear membrane SUN proteins may interact indirectly with lamins, but there was no evidence that they are involved in laminopathies. However, as described herein, loss of the Sun1 gene in Lmna^{-/-} and LmnaΔ9 mice results in extensive rescue of cellular, tissue, organ, and life span abnormalities. In addition, the knock down of over accumulated SUN1 protein in primary HGPS cells corrected their nuclear defects and cellular senescence. The inventors surprisingly discovered Sun1 over accumulation as a potential pivotal pathologic effector of laminopathies.

Based on the above results, the present invention provides a Sun 1 inhibitor for treating a laminopathy in a subject. As used herein the term “inhibitor” or grammatical variation thereof refers to a substance or a compound or an agent capable of delaying, slowing or preventing the activity of a gene product. For example, the present invention provides a substance capable of inhibiting Sun 1 gene expression to reduce the level of Sun 1 gene expression or capable of binding to the expression product of Sun 1 gene to reduce or prevent the activity of Sun 1 gene product.

In the present invention, there is no special limitation on the type of the inhibitors capable of inhibiting Sun 1 gene expression or binding to the expression product of Sun 1 gene in the present invention, as long as it can silence Sun 1 gene expression or inhibit the function of the Sun 1 gene product. It is understood that the inhibitor may be a reversible, quasi-irreversible or irreversible inhibitor. The reversibility of the inhibitor may be determined by method known in the art.

In one example, the inhibitor as disclosed herein include but are not limited to a silencing oligonucleotide, a ribozyme, a Transcription Activator-Like Effector Nuclease (TALEN), a Zinc Finger Nuclease (ZFN), an antibody, an active organic compound and other inhibitors capable of inhibiting Sun 1 gene expression or binding to the expression product of Sun 1 gene.

In another example, the silencing oligonucleotide as disclosed herein include but is not limited to a small interfering RNA (siRNA), a short hairpin RNA (shRNA), a morpholino oligomer, and a micro RNA (miRNA) mimic. The silencing oligonucleotide of the invention is capable of inhibiting expression of Sun 1 gene by interfering with the expression mechanism. For example, inhibition can occur through direct or indirect binding to the genomic region of Sun 1, or interfering with the splicing mechanism of the premRNA of Sun 1, or binding to the mRNA of Sun 1 thereby inhibiting translation to the Sun 1 polypeptide. Other contemplated mechanisms of action of silencing oligonucleotide are well known in the art.

When treating or preventing a laminopathy, the said inhibitor can be one or more small interfering nucleotides, the small interfering nucleotide is a double-strand RNA molecule, including the sense strand and the antisense strand, and the antisense strand of the small interfering nucleotide comprised the region capable of complementing to the mRNA sequence of SUN 1 gene, and the length of the region is less than 30 nucleotides.

For example, the region in the antisense strand of the small interfering nucleotides, which is capable of complementing or is complementary to the mRNA sequence of SUN 1 gene. In one example, the present invention provides a siRNA that may be complementary to the SUN 1 mRNA sequence. The siRNA as disclosed herein may have a nucleotide length ranging from about 8 to 50 nucleotides, usually from about 10 to 50 nucleotides long, more usually from about 20 to 50 nucleotides long, more usually from about 30 to 50 nucleotides long, more usually from about 10 to 40 nucleotides long, more usually from about 10 to 30 nucleotides long, more usually from about 20 to 40 nucleotides long, and more usually from about 30 to 40 nucleotides long. The region in the SUN 1 gene, which is capable of complementing to the antisense strand of the said small interfering nucleotides, is shown as one of SEQ ID Nos: 1-47.

In one example, the nucleotide sequence of said small interfering nucleotide comprises the nucleotide sequence shown as one of SEQ ID Nos 1-47, or the nucleotide sequence of the said small interfering nucleotide comprised modified products of the nucleotide sequence shown as one of SEQ ID Nos 1-47, wherein

| | |
|---|---|
| SEQ ID NO: 1 | 5′-GGUAACUGCUGGGCAUUUA-3′ |
| SEQ ID NO: 2 | 5′-GGUACCAGUUUGUUACUUU-3′ |
| SEQ ID NO: 3 | 5′-GCGCUCAGUUCCAGCUAUU-3′ |
| SEQ ID NO: 4 | 5′-GAAAAGACCCGACGACACA-3′ |
| SEQ ID NO: 5 | 5′-GCACAAACAAAUCAGCUUU-3′ |
| SEQ ID NO: 6 | 5′-GCGCUGUCUCCCUGAAGAA-3′ |
| SEQ ID NO: 7 | 5′-GAACCGAGCGGCCAGAACA-3′ |
| SEQ ID NO: 8 | 5′-CGAGCGGCCAGAACAACAA-3′ |
| SEQ ID NO: 9 | 5′-CCGAGCGGCCAGAACAACA-3′ |
| SEQ ID NO: 10 | 5′-CAGAAGCACAAACAAAUCA-3′ |
| SEQ ID NO: 11 | 5′-AACCGAGCGGCCAGAACAA-3′ |
| SEQ ID NO: 12 | 5′-AGCACAAACAAAUCAGCUU-3′ |
| SEQ ID NO: 13 | 5′-GUAUCAACCACGUGUCAAG-3′ |
| SEQ ID NO: 14 | 5′-CCUGCAGGAUGCUGUGACU-3′ |
| SEQ ID NO: 15 | 5′-CUGUAUUGGACGAGUCUUG-3′ |
| SEQ ID NO: 16 | 5′-CUGAAGAACCGAGCGGCCA-3′ |
| SEQ ID NO: 17 | 5′-UAGUAUCAACCACGUGUCA-3′ |
| SEQ ID NO: 18 | 5′-UUGGACGAGUCUUGGAUUC-3′ |
| SEQ ID NO: 19 | 5′-GAGUCUUGGAUUCGUGAAC-3′ |
| SEQ ID NO: 20 | 5′-CAAAUCAGCUUUUAGUAUC-3′ |
| SEQ ID NO: 21 | 5′-AGUCUUGGAUUCGUGAACA-3′ |
| SEQ ID NO: 22 | 5′-UCAACCACGUGUCAAGGCA-3′ |
| SEQ ID NO: 23 | 5′-UGUCAAGGCAGGUCACGUC-3′ |
| SEQ ID NO: 24 | 5′-AUGGUGAGGCUGUGGGUGC-3′ |
| SEQ ID NO: 25 | 5′-GAGCGGCCAGAACAACAAA-3′ |
| SEQ ID NO: 26 | 5′-GGAUGGUGAGGCUGUGGGU-3′ |
| SEQ ID NO: 27 | 5′-ACUCGACGGCCUCCUGUAU-3′ |
| SEQ ID NO: 28 | 5′-UGAAGAACCGAGCGGCCAG-3′ |
| SEQ ID NO: 29 | 5′-GUCUUGGAUUCGUGAACAG-3′ |
| SEQ ID NO: 30 | 5′-CGUAGUUUGCGCCUGGCCA-3′ |
| SEQ ID NO: 31 | 5′-GCAGGAUGCUGUGACUCGA-3′ |
| SEQ ID NO: 32 | 5′-AGUAUCAACCACGUGUCAA-3′ |
| SEQ ID NO: 33 | 5′-GAAGAACCGAGCGGCCAGA-3′ |
| SEQ ID NO: 34 | 5′-UGCUGUGACUCGACGGCCU-3′ |
| SEQ ID NO: 35 | 5′-UCGACGGCCUCCUGUAUUG-3′ |
| SEQ ID NO: 36 | 5′-GGCCUCCUGUAUUGGACGA-3′ |
| SEQ ID NO: 37 | 5′-AAGCACAAACAAAUCAGCU-3′ |
| SEQ ID NO: 38 | 5′-GUAUUGGACGAGUCUUGGA-3′ |
| SEQ ID NO: 39 | 5′-AGAACCGAGCGGCCAGAAC-3′ |
| SEQ ID NO: 40 | 5′-GCAGCGCUGUCUCCCUGAA-3′ |
| SEQ ID NO: 41 | 5′-GUCACGUCCUCUGGCGUCA-3′ |
| SEQ ID NO: 42 | 5′-CGAGUCUUGGAUUCGUGAA-3′ |
| SEQ ID NO: 43 | 5′-CCUCCUGUAUUGGACGAGU-3′ |
| SEQ ID NO: 44 | 5′-AUUGGACGAGUCUUGGAUU-3′ |
| SEQ ID NO: 45 | 5′-UUGGAUUCGUGAACAGACC-3′ |
| SEQ ID NO: 46 | 5′-GGAUUCGUGAACAGACCAC-3′ |
| SEQ ID NO: 47 | 5′ GUGUCAAGGCAGGUCACGU-3′ |

In the present invention, the said modification may comprise at least one of the modifications as indicated below. In one example, the silencing oligonucleotide may as comprise a chemical modification of one or more nucleotides, which render the silencing oligonucleotide more stable than the non-modified sequence. The chemical modification disclosed herein includes but are not limited to a modification of the phosphate backbone, a modified sugar moiety, a modified nucleotide, and a modified terminal nucleotide.

The modification of the phosphate backbone refers a modification on the phosphodiester bond moiety linking nucleotide in the nucleotide sequence. The said chemical modification is well known to those skilled in the art, the said modifications on phosphodiester bond moiety referred to the substitutions on oxygen in the phosphodiester bond, including sulfur substitution in phosphoric acid moiety and borane substitution in phosphoric acid moiety. These two modifications can stabilize the structure of nucleotide and maintain high specificity and affinity of base group matching.

In one example, the modification of the phosphate backbone disclosed herein includes but is not limited to replacing one or more or all of the phosphate molecules of the nucleotide phosphate backbone with a molecule selected from the group consisting of phosphorothioate, methylphosphonate, phosphotriester, phosphorodithioate and phosphoselenate.

In one example, the modified sugar moiety disclosed herein includes but is not limited to 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine and 2′-amino-butyryl-pyrene-uridine.

In the present invention, the modification of the terminal nucleotide may comprise modification on the 2′-OH of the sugar moiety, for example the ribose moiety in the nucleotide sequence. In one example, the modified terminal nucleotide may have its 2′—OH group substituted with a molecule including but not limited to alkyl, substituted alkyl, alkaryl-, aralkyl-, —F, —Cl, —Br, —CN, —CF₃, —OCF₃, —OCN, —O-alkyl, —S-alkyl, —O-allyl, —S— allyl, HS-alkyl-O, —O-alkenyl, —S-alkenyl, —N-alkenyl, —SO-alkyl, -alkyl-OSH, -alkyl-OH, —O-alkyl-OH, —O-alkyl-SH, —S-alkyl-OH, —S-alkyl-SH, -alkyl-S-alkyl, -alkyl-O-alkyl, —ONO₂, —NO₂, —N₃, —NH₂, alkylamino, dialkylamino-, aminoalkyl-, aminoalkoxy, aminoacid, aminoacyl-, —ONH₂, —O-aminoalkyl, —O-aminoacid, —O-aminoacyl, heterocycloalkyl-, heterocycloalkaryl-, aminoalkylamino-, polyalklylamino-, substituted silyl-, methoxyethyl-(MOE), alkenyl and alkynyl. Example of the modification on 2′-OH in ribose moiety of the nucleotides may be such as modification as 2′-fluor(o) substitution, modification as 2′-oxo-methyl substitution, modification as 2′-oxo-ethidene-methoxyl substitution, modification as 2,4′-dinitrophenol substitution, modification as locked nucleic acid (LNA), modification as 2′-amino substitution, or 2′-deoxy-modification.

In the present invention the modified nucleotide may comprises a modified base. In one example the modified base includes but is not limited to 2-aminoadenosine, 2,6-diaminopurine, inosine, pyridin-4-one, pyridin-2-one, phenyl, pseudouracil, 2,4,6-trimethoxy benzene, 3-methyl uracil, dihydrouridine, naphthyl, aminophenyl, 5-alkylcytidine (e.g., 5-methylcytidine), 5-alkyluridine (e.g., ribothymidine), 5-halouridine (e.g., 5-bromouridine), 6-azapyrimidine, 6-alkylpyrimidine (e.g. 6-methyluridine), propyne, queuosine, 2-thiouridine, 4-thiouridine, wybutosine, wybutoxosine, 4-acetylcytidine, 5-(carboxyhydroxymethyl)uridine, 5-carboxymethylaminomethyl-2-thiouridine, 5-carboxymethylaminomethyluridine, beta-D-galactosylqueuosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueuosine, uridine-5-oxyacetic acid, 2-thiocytidine, 3,N(4)-ethanocytosine, 8-hydroxy-N6-methyladenine, 4-acetylcytosine, 5-fluorouracil, 5-bromouracil, 5-carboxymethylaminomethyl-2-thiouracil, 5-carboxymethylaminomethyluracil, dihydrouracil, N6-isopentyl-adenine, 1-methylpseudouracil, 1-methylguanine, 2,2-dimethylguanine, 2-methylguanine, 3-methylcytosine, N6-methyladenine, 5-methoxy amino methyl-2-thiouracil, β-D-mannosylqueuosine, 5-methoxycarbonylmethyluracil, 2 methylthio-N6-isopentenyladenine, uracil-5-oxyacetic acid methyl ester, pseudouracil, 2-thiocytosine, 5-methyl-2-thiouracil, 2-thiouracil, 4-thiouracil, 5-methyluracil, N-uracil-5-oxyacetic acid methylester, uracil 5-oxyacetic acid, 2-thiocytosine, 5-propyluracil, 5-propylcytosine, 5-ethyluracil, 5-ethylcytosine, 5-butyluracil, 5-pentyluracil, 5-pentylcytosine, 2,6,-diaminopurine, methylpseudouracil, 1-methylguanine and 1-methylcytosine.

Nucleic acids suitable for use in the context of the invention include, but are not limited to, those comprising a nucleic acid sequence containing regions that are at least about 30%, 50%, 60%, 70%, 75%, 80%, 85%, 90%, 95%, 97%, 98% or 99% identical to a region of SEQ ID NOs: 1 to 47 of identical size.

The inventive method is preferably performed as soon as possible after it has been determined that a subject is at risk for developing a laminopathy (e.g., diagnosis of close family member) or as soon as possible after onset of the laminopathy is detected. To this end, Sun 1 is administered before symptoms appear to protect, in whole or in part, against the onset of laminopathy. Sun 1 also can be administered after symptoms are detected to prevent, in whole or in part, additional symptoms or an increase in symptom severity.

A particular administration regimen for a subject will depend, in part, upon the form of Sun 1 administered (e.g., polypeptide or nucleic acid molecule), the amount administered, the route of administration, and the cause and extent of any side effects. The amount of Sun 1 administered to a subject (e.g., a mammal, such as a human) in accordance with the invention should be sufficient to effect the desired response over a reasonable time frame. Dosage typically depends upon a variety of factors, including the particular agent employed, the age and body weight of the subject, as well as the existence of any disease or disorder in the subject. The clinician may titer the dosage and may modify the route of administration to obtain the optimal therapeutic effect, and conventional range-finding techniques are known to those of ordinary skill in the art. Purely by way of illustration, the inventive method can comprise administering, e.g., from about 0.1 μg/kg to up to about 100 mg/kg of Sun 1 or more, depending on the factors mentioned above. In other embodiments, the dosage may range from 1 μg/kg up to about 100 mg/kg; or 5 μg/kg up to about 100 mg/kg; or 10 μg/kg up to about 100 mg/kg. Some conditions or disease states require prolonged treatment, which may or may not entail administering lower doses of agent over multiple administrations. In addition, when appropriate, Sun 1 is administered in combination with other substances (e.g., therapeutics) and/or other therapeutic modalities to achieve an additional (or augmented) biological effect.

To deliver the silencing oligonucleotide to a subject having or suspected to have a laminopathy, the present invention provides for a delivery vehicle to be formulated with said silencing oligonucleotide. The delivery vehicle when formulated with the silencing oligonucleotide may allow delivery of the silencing oligonucleotide to the target site in a patient having or suspected to have a laminopathy. The delivery vehicle may be such that the silencing oligonucleotide is protected from degradation, has an increased half-life, is capable of delivering the silencing oligonucleotide to the Sun 1 target thereby inhibiting the Sun 1 gene. As used in the present disclosure, the term “subject” or “patient” refers to a mammal such as a rodent, cat, dog, primate or human, preferably said subject or patient is a human.

In one example, the delivery vehicle may be a nanoparticle. The nanoparticle of the invention includes but is not limited to a liposome, a peptide, an aptamer, an antibody, a polyconjugate, a microencapsulation, a virus like particle (VLP), a nucleic acid complex and a mixture thereof. For example, the liposome as disclosed herein includes but is not limited to a stable nucleic acid-lipid particle (SNALP), a 1,2-dioleoyl-sn-glycero-3-phosphocholine (DOPC) based delivery system, and a lipoplex. As described herein, the term “liposome” refers to an artificial vesicle composed of one or more concentric phospholipid bilayers and used especially to deliver microscopic substances (as drugs or nucleic acid) to body cells.

The term “aptamer” refers to oligonucleic acid or peptide molecules that bind to a specific molecular target such as small molecules, proteins, nucleic acids, and even cells, tissues and organisms. The term “lipoplex” as used herein refers to non-viral vehicles, such as cationic liposomes and the complexes they form with nucleic acid molecules. Lipoplexes are often presented as the most promising alternative to the use of viral vectors for gene therapy.

Suitable methods of administering a physiologically acceptable composition, such as a pharmaceutical composition comprising a Sun 1 inhibitor, are well known in the art. Although more than one route can be used to administer an agent, a particular route can provide a more immediate and more effective reaction than another route. Depending on the circumstances, a pharmaceutical composition comprising Sun 1 is applied or instilled into body cavities, absorbed through the skin or mucous membranes, ingested, inhaled, and/or introduced into circulation.

In the present invention, the silencing oligonucleotide may be administered by the same or different routes. For example, the silencing oligonucleotide is administered systemically. The present disclosure also envisages administering the silencing oligonucleotide locally.

In some instances, the silencing oligonucleotide may be administered orally, intraadiposally, intraarterially, intraarticularly, intracranially, intradermally, intralesionally, intramuscularly, intranasally, intraocularally, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, orally, parenterally, rectally, subconjunctivally, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof. For example, in some variations, the silencing oligonucleotide is administered intravenously, intra-arterially or orally. For example, in some variations, the silencing oligonucleotide is administered intravenously. In one example, the silencing oligonucleotide as disclosed herein may be formulated for systemic administration. To facilitate administration, a protein or nucleic acid molecule can be formulated into a physiologically-acceptable composition comprising a carrier (i.e., vehicle, adjuvant, or diluent). The particular carrier employed is limited only by chemico-physical considerations, such as solubility and lack of reactivity with the therapeutic, and by the route of administration. Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions. Injectable formulations are further described in the art. A pharmaceutical composition comprising Sun 1 inhibitor may be placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

The pharmaceutically effective amount of the Sun 1 inhibitor to be used for treatment of laminopathy can be a daily dose is 0.01-25 mg of composition per kg of body weight. In some variations, the daily dose is 0.05-20 mg of composition per kg of body weight. In some variations, the daily dose is 0.1-10 mg of composition per kg of body weight, or 1-10 mg of composition per kg of body weight. In some variations, the daily dose is 0.1-5 mg of composition per kg of body weight. In some variations, the daily dose is 0.1-2.5 mg of composition per kg of body weight. In some variations, the daily dose is 0.1-0.24 mg of composition per kg of body weight.

The amount of Sun 1 inhibitor in the formulation can be from about 0.1 mg to about 500 mg. In some variations, the daily dose can be from about 1 mg to about 300 mg. In some variations, the daily dose can be from about 10 mg to about 200 mg of the formulation. In some variations, the daily dose can be about 25 mg of the formulation. In other variations, the daily dose can be about 75 mg of the formulation. In still other variations, the daily dose can be about 150 mg of the formulation. In further variations, the daily dose can be from about 0.1 mg to about 30 mg of the formulation. In some variations, the daily dose can be from about 0.5 mg to about 20 mg of the formulation. In some variations, the daily dose can be from about 1 mg to about 15 mg of the formulation. In some variations, the daily dose can be from about 1 mg to about 10 mg of the formulation. In some variations, the daily dose can be from about 1 mg to about 5 mg of the formulation.

Any laminopathy improved by administration of Sun 1 is suitable for prophylactic or therapeutic treatment by the inventive method. Laminopathies appropriate for treatment include, but are not limited to, Hutchinson-Gilford Progeria syndrome (HGPS), Emery-Dreifuss Muscular Dystrophy (EDMD), cardiomyopathy, Atypical Werner syndrome, Barraquer-Simons syndrome, Buschke-Ollendorff syndrome, Charcot-Marie-Tooth disease, Familial partial lipodystrophy of the Dunnigan type (FPLD), Greenberg dysplasia, Leukodystrophy, Limb-girdle muscular dystrophy type 1B, Lipoatrophy with diabetes, hepatic steatosis, hypertrophic cardiomyopathy, and leukomelanodermic papules (LDHCP), Mandibuloacral dysplasia with type A lipodystrophy (MADA), Mandibuloacral dysplasia with type B lipodystrophy (MADB), Pelger-Huet anomaly (PHA), Pelizaeus-Merzbacher disease and Tight skin contracture syndrome

In some examples, the laminopathy may be such as laminopathic lipodystrophy disorders, systemic laminopathies, laminopathic neurological disorders, or muscle laminopathies. By “laminopathic” lipodystrophy disorders and “laminopathic” neurological disorders is meant lypodystrophy and neurological disorders resulting from or associated with abnormal nuclear envelope morphology. Lipodystrophy disorders are characterized by abnormal distribution of adipose tissue, optionally associated with metabolic disorders such as diabetes and hypertriglyceridemia. Lipodystrophy patients often experience selective loss and/or excessive accumulation of adipose tissue in certain regions of the body (e.g., loss in the limbs accompanied by excessive deposit in the upper back). Examples of laminopathic lipodystrophy disorders include, for instance, familial partial lipodystrophy (Dunnigan type), acquired partial lipodystrophy, type A insulin resistance syndrome, generalized lipoatrophy syndrome, and familial partial lipodystrophy (Kobberling).

Systemic laminopathies affect a variety of tissue types and include, e.g., atypical Werner syndrome, progeria (e.g., Hutchinson-Gilford progeria syndrome), restrictive dermopathy, and mandibuloacral dysplasia. The symptoms associated with systemic laminopathies are diverse. Atypical Werner syndrome patients prematurely exhibit features commonly associated with aging such as short stature, osteoporosis, thinning hair, athlerosclerosis, and cataracts. Restrictive dermopathy, on the other hand, is commonly associated with skin and joint contracture, abnormal skull mineralization, and pulmonary defects. Laminopathic neurological disorders, or laminopathies with peripheral nerve involvement, also are suitable for treatment by the inventive method. Neurological laminopathies include, e.g., Charcot-Marie-Tooth disease type 2B1, autosomal dominant leukodystrophy, and autosomal dominant spinal muscular dystrophy.

A majority of laminopathies caused by lamin A/C mutations involve striated muscle. Emery-Dreifuss muscular dystrophy (EDMD), limb-girdle muscular dystrophy type 1B, congenital muscular dystrophy, multisystem dystrophy syndrome, dilated cardiomyopathy 1A, and dilated cardiomyopathy with conduction system defects are diagnosed as muscle laminopathies. Patients suffering from muscle laminopathies exhibit, for example, muscle weakness or wasting, hypertrophy of select muscles (e.g., calf), muscle or tendon contractures, cardiomyopathy, impaired cardiac conduction, and mental retardation.

The present invention also provides a method of diagnosing a laminopathy, or determining if an individual is at risk of developing a laminopathy. The method may measuring the expression level of Sun1 in an individual or a sample obtained from the individual and comparing the Sun1 expression levels obtained from the step of measuring described above with a control reference. In the method described, an elevated level of Sun1 in the individual compared to the control indicates that the individual has a laminopathy or is at risk of developing a laminopathy.

The laminopathy may be a laminopathic lipodystrophy disorder, a systemic laminopathy, or a laminopathic neurological disorder. In a specific aspect of the invention, the laminopathy is a muscle laminopathy (e.g., Emery-Dreifuss muscular dystrophy (such as Emery-Dreifuss muscular dystrophy type 2), limb-girdle muscular dystrophy type 1B, congenital muscular dystrophy, multisystem dystrophy syndrome, dilated cardiomyopathy 1A, or dilated cardiomyopathy with conduction system defects). While detection of mutant Sun 1 may not, by itself, absolutely predict development of a particular disease, the presence or absence of Sun 1 mutants indicates an increased and/or decreased likelihood that a subject will develop symptoms associated with a laminopathy. This information is extremely valuable, and allows a subject to perform regular physical exams to monitor the progress and/or appearance of symptoms at an early stage.

The diagnostic method entails detecting measuring expression level of Sun 1 in a biological sample from a subject. Numerous methods of obtaining subject samples are widely used in the art and are appropriate in the context of the invention. Samples typically are isolated from blood, serum, urine, amniotic fluid, or tissue biopsies from, e.g., muscle, connective tissue, nerve tissue, placenta, and the like. If the subject is a fetus, a sample can be obtained by amniocentesis or chorionic villus sampling. Once obtained, cells from the sample are examined to detect the presence or absence of Sun 1, and its expression level.

It will be appreciated that Sun 1 can be detected in a variety of ways. In one example, the method comprises obtaining nucleic acid sequence data from the cellular sample. Suitable methods of directly analyzing a nucleic acid molecule include, for instance, denaturing high pressure liquid chromatography (DHPLC), DNA hybridization, computational analysis, automated fluorescent sequencing, clamped denaturing gel electrophoresis (CDGE), denaturing gradient gel electrophoresis (DGGE), mobility shift analysis, restriction enzyme analysis, heteroduplex analysis, chemical mismatch cleavage (CMC), RNase protection assays, use of polypeptides that recognize nucleotide mismatches, and direct manual sequencing. These and other methods are described in the art.

In one embodiment, diagnosis of (or identification of a predisposition to) laminopathy can be accomplished using a hybridization method. A biological sample of genomic DNA, RNA, or cDNA is obtained from a subject suspected of having, being susceptible to, or experiencing symptoms associated with laminopathy. Optionally, the nucleic acid encoding Sun 1 is amplified by polymerase chain reaction (PCR). The DNA, RNA, or cDNA sample is then, examined. The presence of Sun 1 can be determined by sequence-specific hybridization of a nucleic acid probe specific for particular mutation within the Sun 1 coding sequence. As discussed above, a nucleic acid probe is a DNA molecule or an RNA molecule that hybridizes to a complementary sequence in genomic DNA, RNA, or cDNA. In some aspects, the presence of more than one Sun 1 mutation is determined by using multiple nucleic acid probes, each being specific for a particular mutation.

One of skill in the art has the requisite knowledge and skill to design a probe so that sequence-specific hybridization will occur only if a particular mutation is present in a Sun 1 coding sequence. By “sequence-specific hybridization” is meant that the probe(s) preferentially bind to a nucleic acid sequence encoding Sun 1. In some embodiments, specific hybridization is achieved using “stringent conditions,” which are conditions for hybridization and washing under which nucleotide sequences at least 60% identical to each other typically remain hybridized. It is appreciated in the art that stringent conditions can differ depending on sequence content, probe length, and the like. Generally, stringent conditions are selected to be about 5° C. lower than the thermal melting point (Tm) for a specific sequence at a defined ionic strength and pH. Tm is the temperature (under defined ionic strength, pH, and nucleic acid concentration) at which 50% of the probes complementary to the target sequence hybridize to the target sequence at equilibrium. Since target sequences are generally present at excess, 50% of the probes are occupied at equilibrium at Tm. Stringent conditions also may include a salt concentration less than about 1.0 M sodium ion, typically about 0.01 to 1.0 M sodium ion (or other salts) at pH 7.0 to 8.3 and the temperature is at least about 30° C. for short probes, primers, or oligonucleotides (e.g., 10 nucleotides to 50 nucleotides) and at least about 60° C. for longer probes, primers and oligonucleotides. Stringent conditions may also be achieved with the addition of destabilizing agents, such as formamide. A non-limiting example of stringent hybridization conditions are hybridization in a high salt buffer comprising 6×SSC, 50 mM Tr-is-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C.

Specific hybridization, if present, is detected using standard methods. For example, the probe can comprise a fluorescent moiety at its 3′ terminus, a quencher at its 5′ terminus, and an enhancer oligonucleotide to facilitate detection. In this detection method, an enzyme cleaves the fluorescent moeity from a fully complementary detection probe, but does not cleave the fluorescent moeity if the probe contains a mismatch. The presence of a particular target sequence is signalled by the fluorescence of the released fluorescent moiety. Alternatively, nucleic acids encoding Sun 1 are dot-blotted using standard methods, and the blot is contacted with one or more oligonucleotide probes specific for a Sun 1 mutation. Similarly, arrays of oligonucleotide probes complementary to target nucleic acid sequence(s) can be employed in the inventive diagnostic method. Oligonucleotide arrays typically comprise a plurality of different oligonucleotide probes coupled to a surface of a substrate (e.g., plastic, complex carbohydrate, or acrylic resin) in different known locations. Such arrays are generally produced using mechanical synthesis methods or light-directed synthesis methods, although other methods are known to the ordinary skilled practitioner.

In another hybridization method, Northern analysis is used to identify the presence of Sun 1 encoded by mRNA in a subject's sample. Specific hybridization between the nucleic acid probe and the nucleic acid in the subject sample indicates that Sun 1 is present, and the subject is suffering from or is at risk of developing a laminopathy.

Sequence analysis can also be used to detect specific Sun 1 mutations associated with laminopathy. Therefore, in one embodiment, determination of the presence or absence of mutant Sun 1 entails directly sequencing DNA or RNA obtained from a subject. If desired, PCR is used to amplify a portion of a nucleic acid encoding Sun 1, and the presence of a specific mutation is detected directly by sequencing the relevant site(s) of the DNA or RNA in the sample.

Mutations in the Sun 1 coding sequence may lead to altered expression levels, e.g., a decrease in the expression level of an mRNA or protein, which lead to an abnormal phenotype. Such mutations are detected via, e.g., ELISA, radioimmunoassays, immunofluorescence, Northern blotting, and Western blotting to compare Sun 1 expression levels in a subject compared to a biologically-matched control or reference. These processes are described in the art.

Alternatively or in addition, the diagnostic method entails detecting variant SUN 1 protein comprising an altered amino acid sequence (e.g., one or more deletions, substitutions, additions, and/or truncation) compared to wild-type SUN 1. Any method of detecting mutant proteins is appropriate for use in the context of the invention, and many are known in the art. For example, Sun 1 may be isolated from a cellular sample and subjected to amino acid sequencing, the results of which are compared to a reference amino acid sequence. Mutant Sun 1 also can be identified by detecting altered molecular weights compared to wild-type Sun 1 using gel electrophoresis (e.g., SDS-PAGE). Immunoassays, e.g., immunofluorescent immunoassays, immunoprecipitations, radioimmunoasays, ELISA, and Western blotting, also can be used.

Several detection methods are accomplished using an anti-Sun 1 antibody or fragment thereof that selectively (or preferentially) binds mutant Sun 1. The term “antibody” refers to a complete (intact) antibody (immunoglobulin) molecule (including polyclonal, monoclonal, chimeric, humanized, or human versions having full length heavy and/or light chains) or a Sun 1 binding fragment thereof. Antibody fragments include F(ab′)2, Fab, Fab′, Fv, Fc, and Fd fragments, and can be incorporated into single domain antibodies, single-chain antibodies, maxibodies, minibodies, intrabodies, diabodies, triabodies, tetrabodies, v-NAR and bis-scFv.

The term “selectively binds” refers to the ability of the antibody or fragment thereof to bind to mutant Sun 1 with greater affinity (e.g., at least 10, 15, 20, 25, 50, 100, 250, 500, 1000, or 10,000 times greater affinity) than it binds to an unrelated control protein, such as hen egg white lysozyme. Preferably, the antibody distinguishes mutant Sun 1 from wild-type Sun 1. Binding affinity can be determined using any of a number of methods known in the art such as an affinity ELISA assay, a BIAcore assay (i.e., a surface plasmon resonance-based assay), a kinetic method, or an equilibrium/solution method.

Various procedures known within the art may be used for the production of antibodies to a mutant Sun 1 protein. For example, monoclonal antibodies that bind to specific antigens may be obtained via the methods described in the art.

Antibody fragments may be derived from intact antibodies using any suitable standard technique such as proteolytic digestion, or optionally, by proteolytic digestion (for example, using papain or pepsin) followed by mild reduction of disulfide bonds and alkylation. Alternatively, such fragments may also be generated by recombinant genetic engineering techniques, such as those techniques known in the art.

In certain aspects, the mutant Sun 1 is identified by detecting changes in function or activity compared to wild-type Sun 1. In this regard, impaired binding to lamin A/C, reduced ability to mediate organized nuclear envelopes, misshapen and herniated nuclei, reduced localization to the nucleus, and/or regions of nuclear envelope pile-up suggest the presence of mutant Sun 1. Methods of detecting binding activity include, for example, competitive binding assays; quantitative binding assays using instruments such as, for example, a Biacore® 3000 instrument; and chromatographic assays, e.g., HPLC and TLC.

The present invention also provides a method of monitoring the progression or treatment of a laminopathy. The method may comprise measuring the expression level of Sun1 in an individual or a sample obtained from the individual and comparing the Sun1 expression levels obtained from above with a control reference wherein an elevated level of Sun1 in the individual compared to the control indicates that the laminopathy has progressed from a less advanced stage to a more advanced stage.

The method described herein may be useful for the diagnosis and/or the monitoring of the progression of laminopathies as disclosed herein.

### Examples

Non-limiting examples of the invention and a comparative example will be further described in greater detail by reference to specific Examples, which should not be construed as in any way limiting the scope of the invention.

Loss of Sun1 Ameliorates Lmna^{-/-} and LmnaΔ9 Pathologies

To gain insight into the co-operativity, if any, between inner nuclear membrane (INM) proteins and the underlying lamina in disease development, the inventors bred Sun1^{+/-} and Lmna^{+/-} mice to produce Lmna^{-/-}Sun1^{-/-} offspring. In view of previous disclosures, it was anticipated that inactivating both the Lmna and Sun1 genes in Lmna^{-/-}Sun1^{-/-} mice would lead to a more severe pathological phenotype than that seen for Lmna^{-/-} animals.

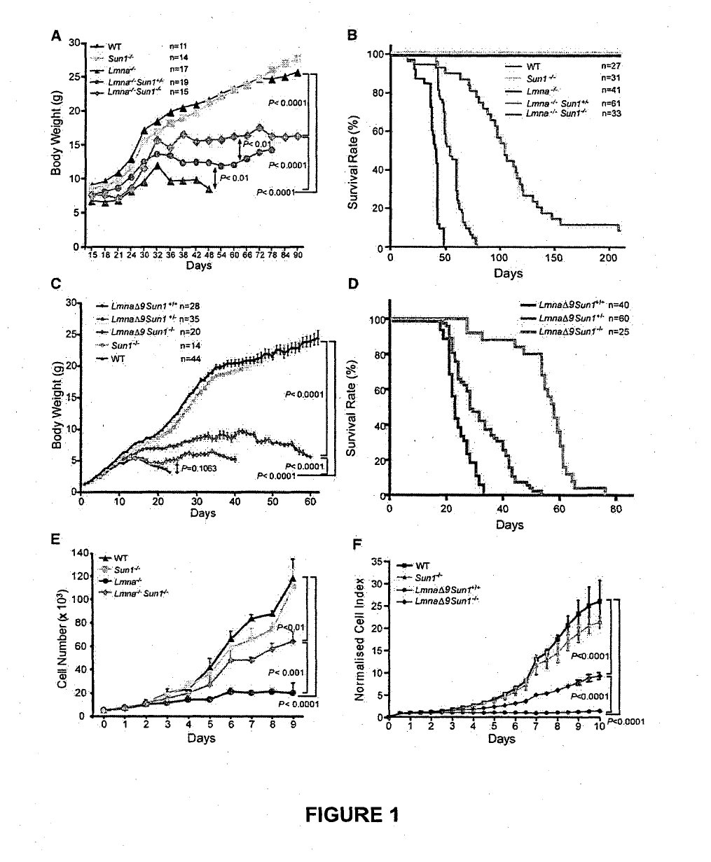

Surprisingly, the inventors observed the opposite. In the Lmna^{-/-} context, the removal of Sun1, rather than exacerbating pathology, unexpectedly ameliorated deficits in body weight (FIG. 1A; P<0.0001), and longevity (FIG. 1B; P<0.01). This rescue of Lmna^{-/-} mice by loss of Sun1 was verified in a second mouse laminopathy model, the LmnaΔ9 mutant.

The body weight and longevity deficits in LmnaΔ9 mice were also rescued in its LmnaΔ9Sun1^{-/-} counterparts (FIG. 1C, D). Remarkably, while all LmnaΔ9 mice expired by 30 days after birth, their LmnaΔ9Sun1^{-/-} littermates thrived past this date, most achieving lifespans more than twice this duration (FIG. 1D). At the cellular level, the severely reduced proliferation of Lmna^{-/-} and LmnaΔ9 fibroblasts was also substantially corrected in Lmna^{-/-}Sun1^{-/-} and LmnaΔ9Sun1^{-/-} cells (FIG. 1E, F).

Tissue Pathologies of Lmna^{-/-} Mice are Improved in Sun1^{-/-}Lmna^{-/-} Mice

Lmna^{-/-} and Lmna^{-/-}Sun1^{-/-} animals grow better and live longer than their corresponding LmnaΔ9 and LmnaΔ9Sun1^{-/-} counterparts (FIG. 1A-D). Cultured Lmna^{-/-} and Lmna^{-/-}Sun1^{-/-} cells proliferated well while LmnaΔ9 and LmnaΔ9Sun1^{-/-} cells are challenging, requiring extracellular matrices or hypoxic conditions for propagation. For further detailed characterizations, the inventors studied the Lmna^{-/-} and Lmna^{-/-}Sun1^{-/-} animals and their cells.

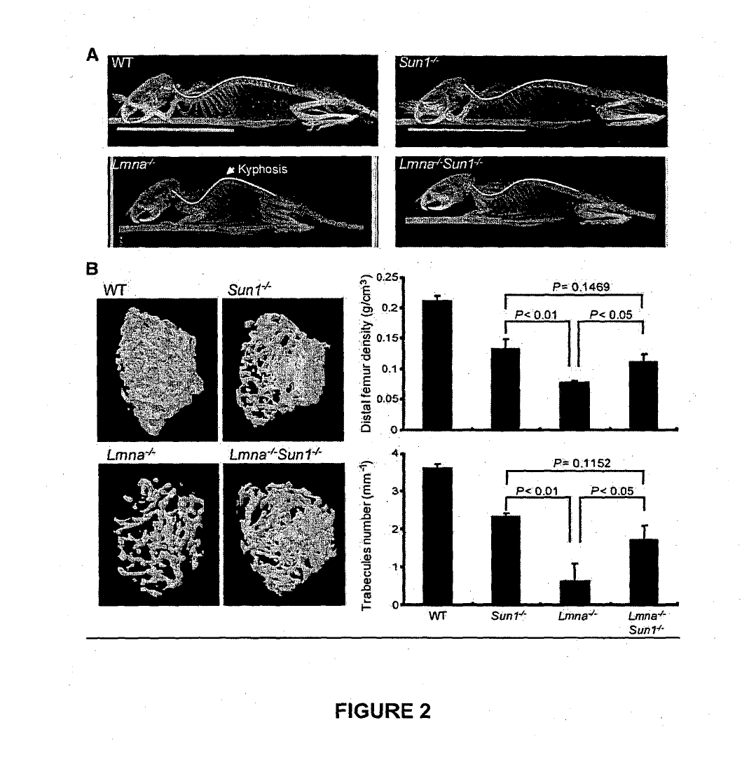

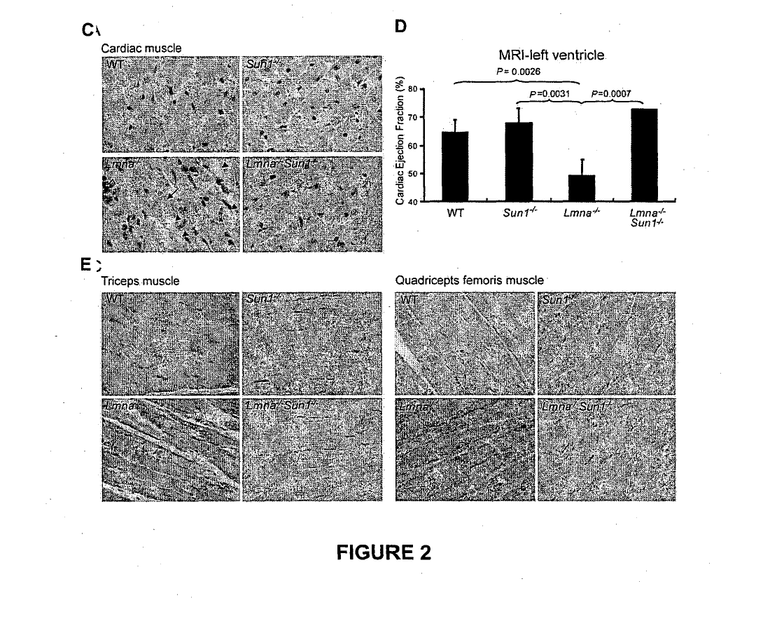

The inventors compared tissue changes in Lmna^{-/-} to Sun1^{-/-}Lmna^{-/-} mice. The spine of Lmna^{-/-} mice by microcomputerized tomography was grossly lordokyphotic; this defect was absent in WT and Sun1^{-/-} mice and was corrected in Lmna^{-/-}Sun1^{-/-} animals (FIG. 2A). The femoral bone of 40-day-old Lmna^{-/-} mice showed trabeculae and bone densities that were notably sparser and thinner than Sun1^{-/-} or WT mice; in Lmna^{-/-}Sun1^{-/-} animals the deficits were markedly improved (FIG. 2B). In other tissues such as cardiac muscle, skeletal muscle, and thyroid glands, pathological changes previously described in the Lmna^{-/-} mice were corrected and improved in the Lmna^{-/-}Sun1^{-/-} mice (FIG. 2 C to E).

Sun1 Accumulates at the Nuclear Envelope (NE) and the Golgi of Lmna^{-/-} MEFs

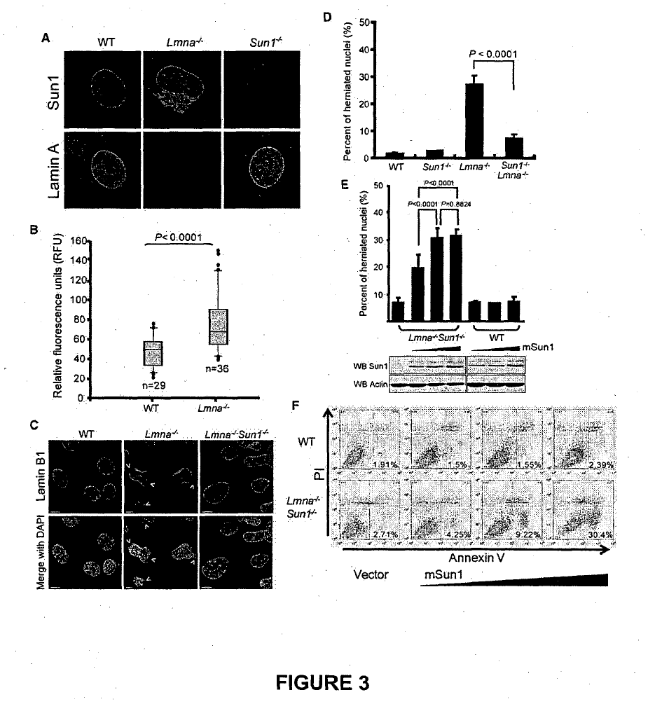

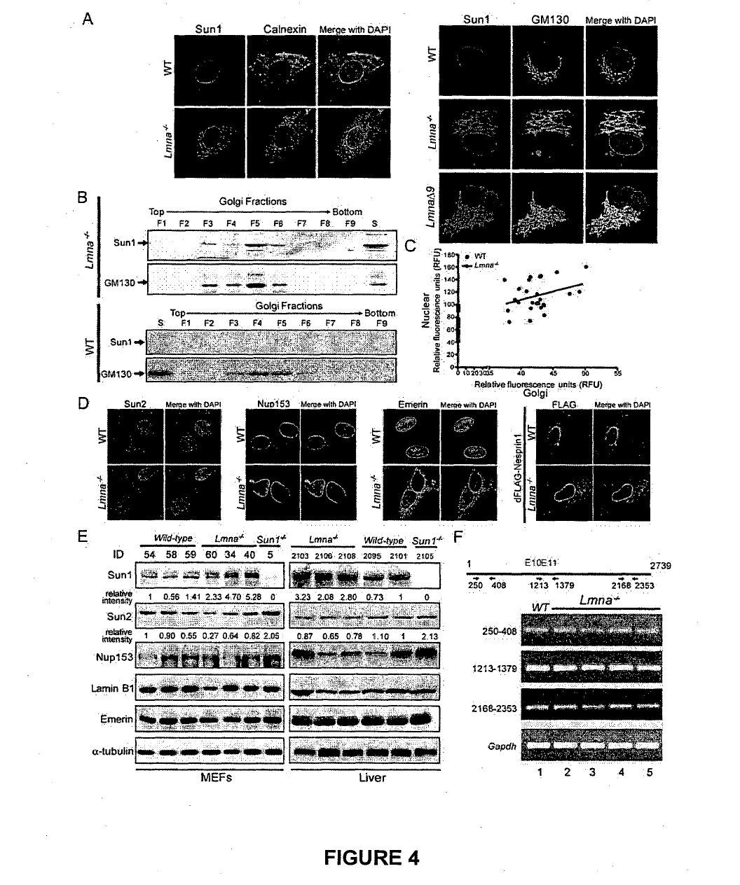

To seek a molecular explanation for loss-of-lamin A changes and their correction by Sun1 depletion, we investigated Sun1 expression in lamin A (WT) and lamin A deficient (Lmna^{-/-}) MEFs. Sun1 and lamin A co-localize at the NE in WT MEFs (FIG. 3A, left panels). By contrast in Lmna^{-/-} MEFs, Sun1 is found in the nuclear envelop (NE) and in increased levels in the Golgi (FIG. 3A, middle panels; and FIG. 4A), based on co-staining with Golgi marker GM130 (FIG. 4A, right) but not with ER marker calnexin (FIG. 4A, left). NE localization and Golgi over accumulation of Sun1 were also seen in LmnaΔ9 mouse fibroblasts (FIG. 4A, right).

That Sun1 localizes with Golgi constituents in Lmna^{-/-} cells was supported by biochemical fractionation of mouse tissue that detected Sun1 and GM130 in the same sucrose density fractions (FIG. 4B). When Lmna^{-/-} cells were examined for the relative distribution of Sun1 in the NE versus the Golgi, the amount in the latter increased proportionally with its level in the former (FIG. 4C), suggesting that over expressed Sun1 protein, in a Lmna^{-/-} context, first occupies and saturates NE sites before “spilling” into the Golgi compartment.

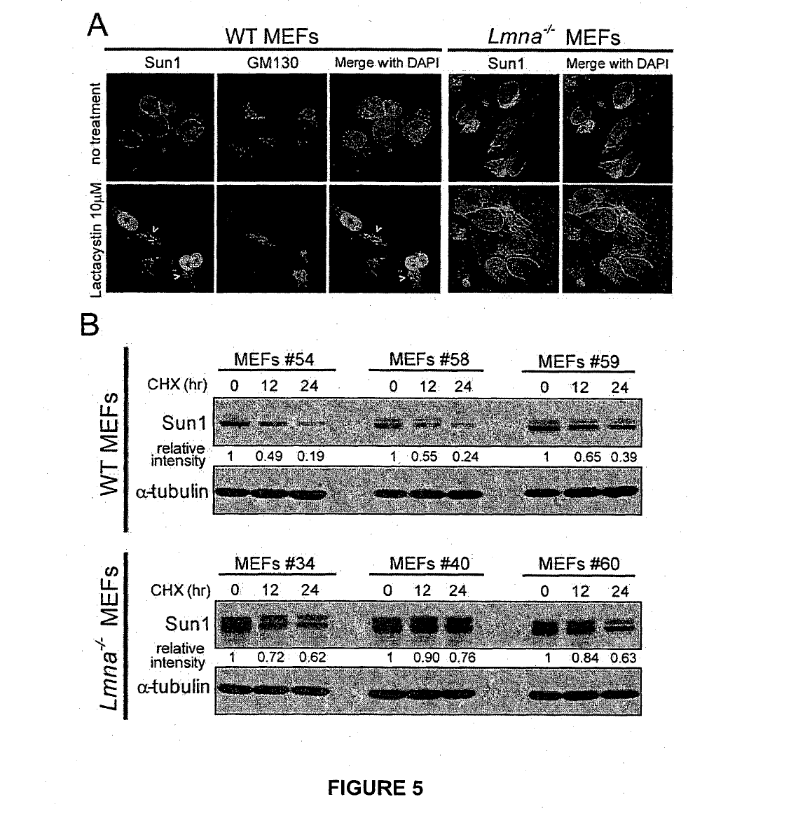

The average Sun1 expression level in individual Lmna^{-/-} MEFs was significantly higher than that in WT MEFs (FIG. 3B, Lmna^{-/-} n=36, WT n=29, P<0.0001) with the highest expressing former cells having approximately 8 fold greater levels of Sun1 than the lowest expressing latter counterparts; by contrast, in Lmna^{-/-} cells other NE proteins such as Sun2 and Nup153 were unchanged in distribution or amounts while Emerin and Nesprin1 were not significantly increased but showed modest increases in cytoplasmic distribution (FIG. 4D, E). The increase in Sun1 protein (FIG. 4E) was not due to elevated Sun1 mRNA levels (compare WT and Lmna^{-/-}; FIG. 4F); this result together with heightened Sun1 accumulation (FIG. 5A) when WT and Lmna^{-/-} MEFs were treated with proteasome inhibitor lactacystin and the prolonged half-life of Sun1 protein in Lmna^{-/-} vs. WT MEFs (FIG. 5B) suggest that Sun1 over accumulation in Lmna^{-/-} cells is due to by reduced protein turnover.

Sun1 Over Accumulation Increases Nuclear Defects

WT MEFs have circular or slightly ovoid nuclei while Lmna^{-/-} nuclei are irregularly shaped with frequent herniations and blebs (FIG. 3C). Intriguingly, the Lmna^{-/-} nuclear abnormalities are significantly (P<0.0001) reduced in Lmna^{-/-}Sun1^{-/-} cells (FIG. 3C, D) suggesting that the nuclear irregularities are not explained simply by loss-of-lamin A which is equally absent in Lmna^{-/-} and Lmna^{-/-}Sun1^{-/-} cells. On the other hand, because both Lmna^{-/-} and LmnaΔ9 cells show Sun1 accumulation in the Golgi (FIG. 3A; FIG. 4A), this event could possibly account for the observed pathologies. This view, if correct, provides a parsimonious explanation for why Lmna^{-/-} and LmnaΔ9 diseases in mice are alleviated when Sun1 levels are reduced (FIG. 1).

The above reasoning predicts that deliberate Sun1 over expression in a Lmna^{-/-} context should exacerbate nuclear aberrancies. To test this, we transfected increasing amounts of a mouse Sun1 (mSun1) expression vector into either Lmna^{-/-}Sun1^{-/-} or WT MEFs.

The over expression of Sun1 progressively increased the prevalence of nuclear herniations in Lmna^{-/-}Sun1^{-/-} MEFs, without significantly affecting WT MEFs (FIG. 3E). The transfections also elicited dose-dependent increases in the apoptosis of Lmna^{-/-}Sun1^{-/-} cells (FIG. 3F).

Golgi-Targeting of Sun1 Elicits Nuclear Herniations

A remarkable feature of Sun1 expression in Lmna^{-/-} MEFs is its mis-accumulation in the extranuclear Golgi apparatus (FIG. 3A; FIG. 4A). Protein mis-accumulation in human organelle storage disorders have been described for lysosomal storage diseases such as Fabry, Tay-Sachs, Gaucher, Niemann-Pick, Pompe, and Krabbe, and endoplasmic reticulum storage diseases such as cystic fibrosis, al-antitrypsin deficiency, hereditary hypoparathyroidism, and procollagen type I, II, IV deficiency; however, to date, there are no good examples of Golgi storage diseases. To test if the deliberate Golgi-mis-accumulation of Sun1 is significantly pathogenic, an HA-tagged Tgn38-fused Golgi-targeting mSun1 expression vector was constructed [Tgn38 is an integral Golgi protein].

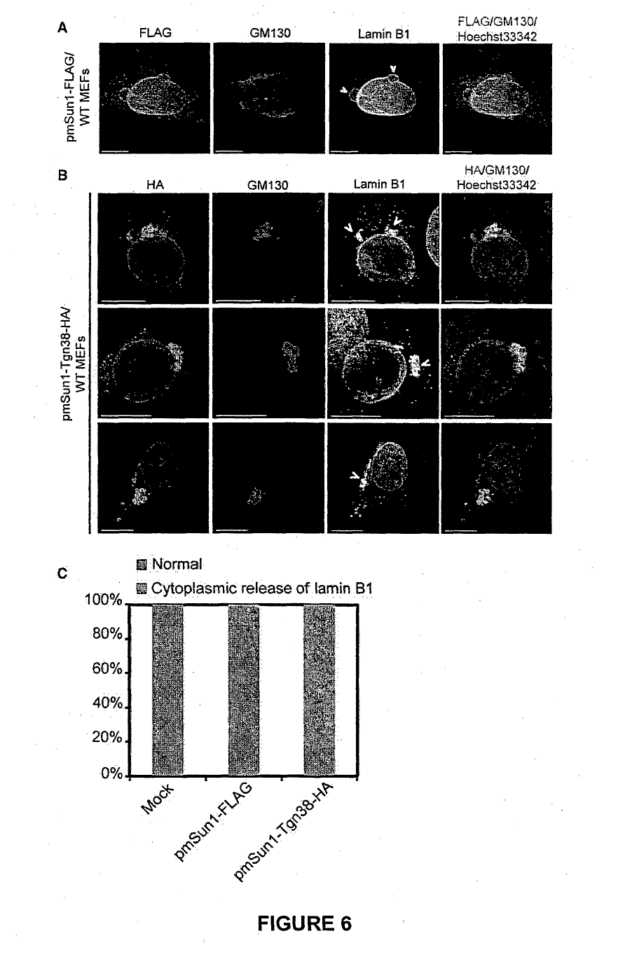

Sun1 protein, when over expressed, in WT MEFs, localized to the nuclear envelope and elicited barely discernable mild nuclear blebbings (FIG. 6A), while transfected Tgn38-Golgi-targeted mSun1 dramatically increased Golgi-accumulation and nuclear herniations with obvious cytoplasmic accumulation of lamin B1 (FIG. 6B) in 83% of Tgn38-Golgi-mSun1 expressing cells (FIG. 6C). Recently, it was reported that the Sun1-related Sun2 protein is physiologically present in the Golgi via a Golgi-retrieval sequence.

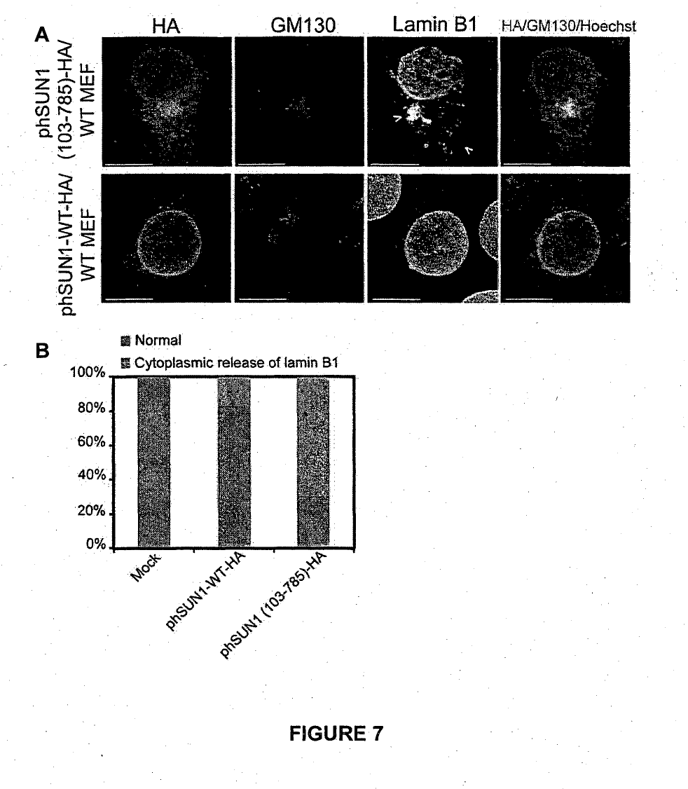

Although not yet determined experimentally, Sun1 may also have a Golgi-locating sequence which could explain why a SUN1-mutant (human SUN1 a.a. 103-785) [FIG. 7] and a wild type Sun1 protein that is expressed in the absence of cell endogenous lamin A (i.e. Lmna^{-/-} cells; FIG. 3A, FIG. 5A), are both found in the Golgi. The inventors also checked if the Golgi-localizing SUN1 (103-785) mutant elicits nuclear aberrations. Unexpectedly, over-expression of the SUN1 (103-785) mutant increased nuclear envelope rupture and redistribution of lamin B1 to the cytoplasm (FIG. 7).

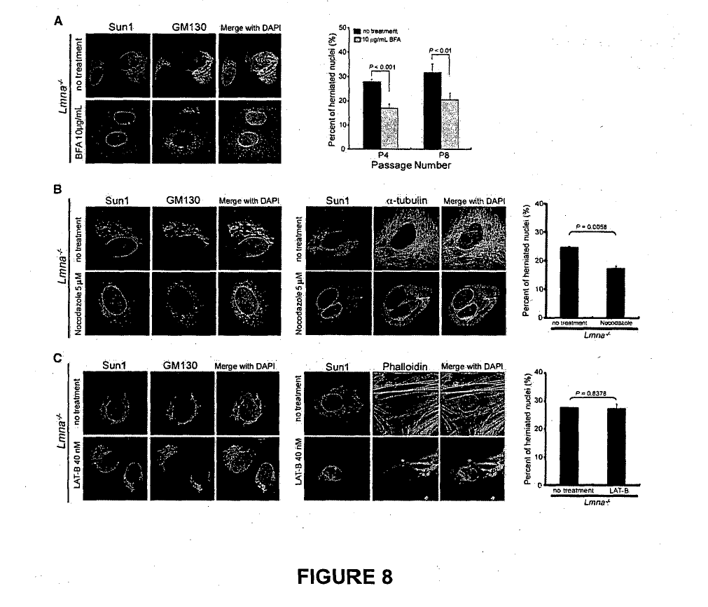

The above results raised the notion that reducing Sun1 accumulation in the Golgi might moderate Lmna^{-/-} nuclear irregularities. Brefeldin A (BFA) is an antibiotic that reversibly interferes with the anterograde transport of macromolecules from the endoplasmic reticulum (ER) to the Golgi. The inventors asked if BFA treatment of Lmna^{-/-} cells would reduce the amount of Sun1 in the Golgi. Confocal imaging of Lmna^{-/-} MEFs treated with BFA at 10 μg/mL for 24 hours showed a reduction in most, albeit not all, Golgi-trafficked Sun1 and GM130 proteins (FIG. 8A, left panels) with statistically significant (P<0.001; P<0.01), reduction in nuclear aberrations in cells passaged four (P4) to eight (P8) times in culture (FIG. 8A, right graph).

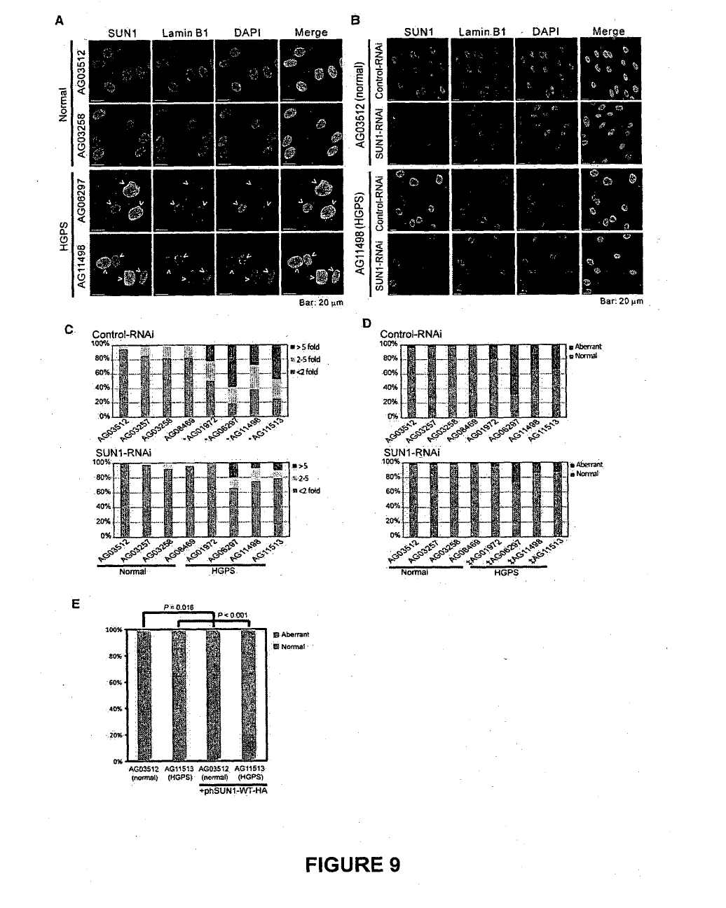

Lmna^{-/-} MEFs were also treated with nocodazole to block microtubule organization (FIG. 8B), or latrunculin B to interrupt actin assembly (FIG. 8C). Nocodazole disrupts the Golgi apparatus, and its treatment of Lmna^{-/-} MEFs indeed led to a punctated redistribution of otherwise Golgi-associated Sun1 and GM130 (FIG. 8B). This treatment also led to a moderate, but statistically significant, reduction of nuclear aberrations (FIG. 9B, right graph). By contrast, latrunculin B did not affect Sun1 distribution in the Golgi and did not ameliorate nuclear defects (FIG. 8C). Collectively, the inventors unexpectedly demonstrated that endogenous (FIG. 8) or exogenous (FIG. 6, 7) Sun1 mis-accumulation in the Golgi elicits substantial cellular pathologies, and reducing Sun1 accumulation in the Golgi restores cellular normalcy.

SUN1 Over Accumulation in HGPS Cells Correlates with Dysfunction

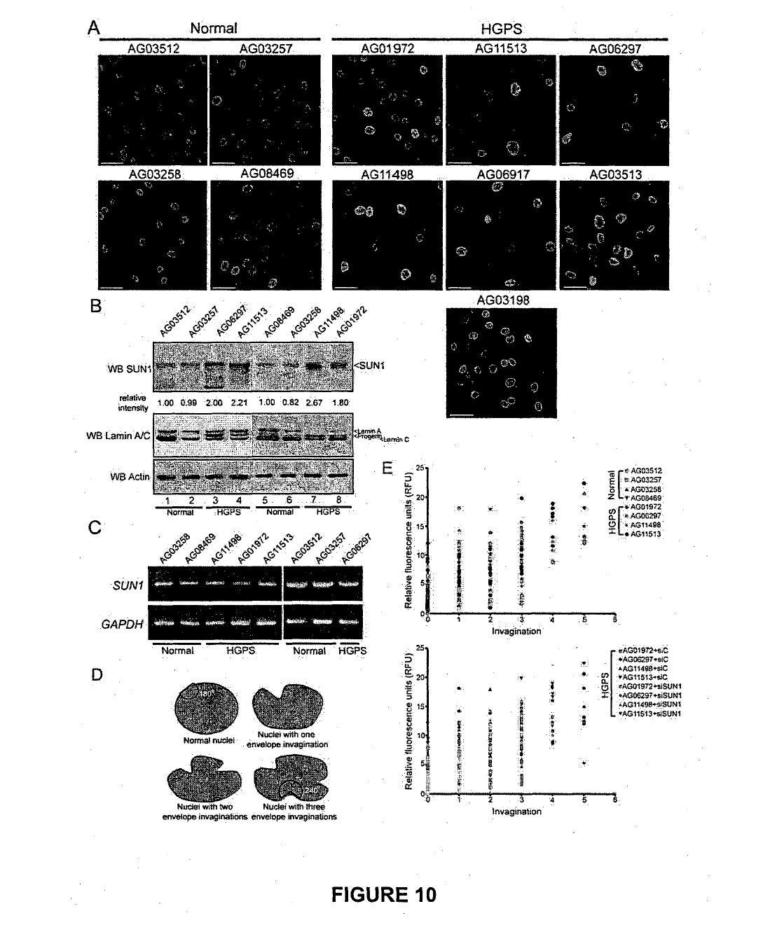

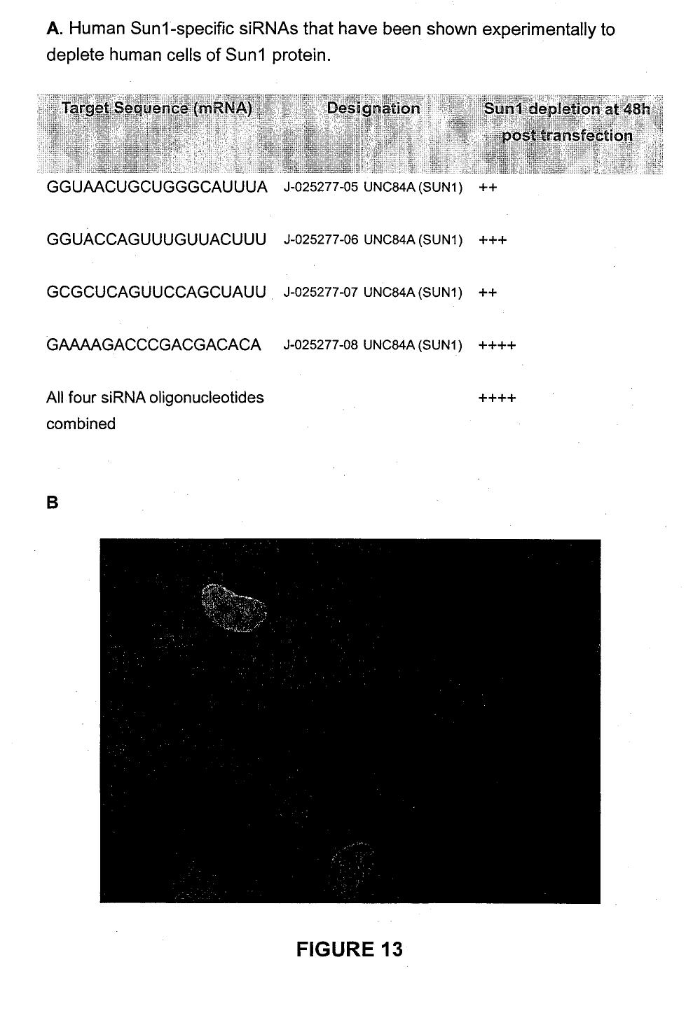

The inventors next investigated SUN1 expression in HGPS cells querying if (and how) this protein might contribute to pathology. SUN1 expression was immunostained in human skin fibroblasts from seven independent HGPS [LMNA 1824C>T (G608G)](FIG. 10A and FIG. 13 A) and four normal control individuals; and verified LAΔ50 progerin expression in HGPS, but not normal cells (FIG. 10B). By immunofluorescence, brighter SUN1 staining was observed in the HGPS (LMNA 1824C>T) cells compared to control cells (representative examples are in FIGS. 9A and 10A, Normal vs. HGPS) which is consistent with increased SUN1 expression by Western blotting (FIG. 10B) and with an earlier report of SUN1 accumulation in HGPS cells. Of note, the stainings showed that not every HGPS cell had elevated SUN1, but cells that stained brightest for SUN1 were also ones that had larger nuclei and more severe nuclear morphological distortions (compare dim-SUN1 HGPS cells, white arrow heads to bright-SUN1 HGPS cells, yellow arrow heads; FIG. 9A). We also determined that SUN1 mRNA levels did not differ significantly in HGPS versus normal cells (FIG. 10C), supporting the interpretation that reduced protein turnover (FIG. 5B), not increased transcription, underlies SUN1 accumulation.

To address if elevated SUN1 levels in HGPS results in pathological defects, we asked if knocking down SUN1 alleviates nuclear defects. SUN1-specific or control siRNAs were transfected into HGPS or normal skin fibroblasts, and nuclear appearance (FIG. 10D) monitored. The nuclear morphologies were unchanged in cells treated with control siRNA (FIG. 9B, 10E); but SUN1-specific siRNA reduced the prevalence of bright-SUN1 HGPS cells (compare AG11498 upper to lower row, FIG. 10B, C), and at the same time lowered the number of cells with aberrant nuclei (FIG. 9B, 9D, 10, 13). The contribution of SUN1 to nuclear morphology was conversely assessed by deliberately over expressing exogenous SUN1. Here, ectopic over expression of SUN1 in HGPS and normal skin fibroblasts significantly increased the prevalence of aberrant nuclei (FIG. 9E).

SUN1 Expression Correlates with HGPS Heterochromatin Profile and Cellular senescence

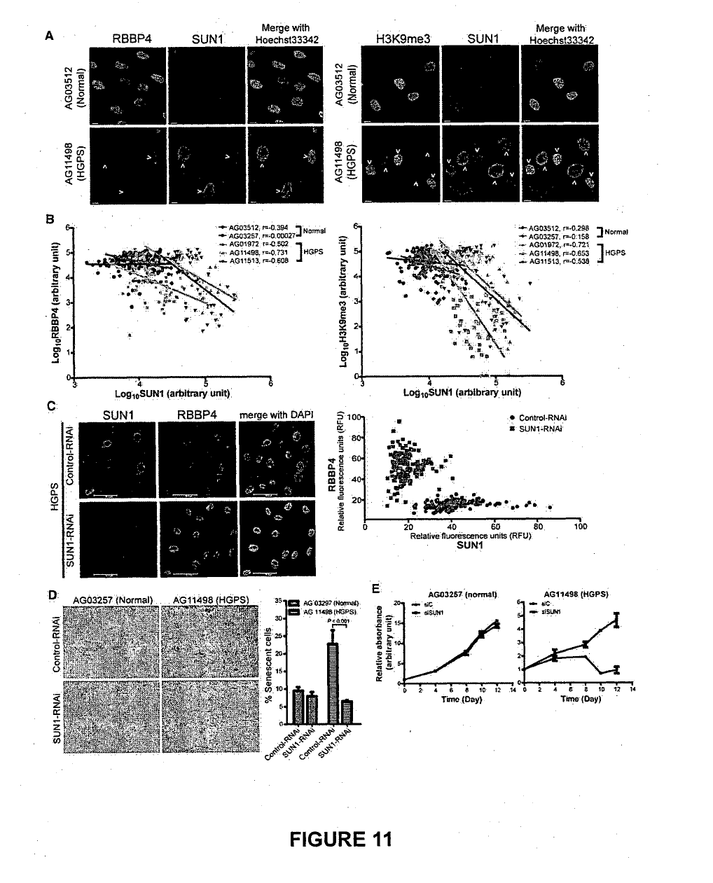

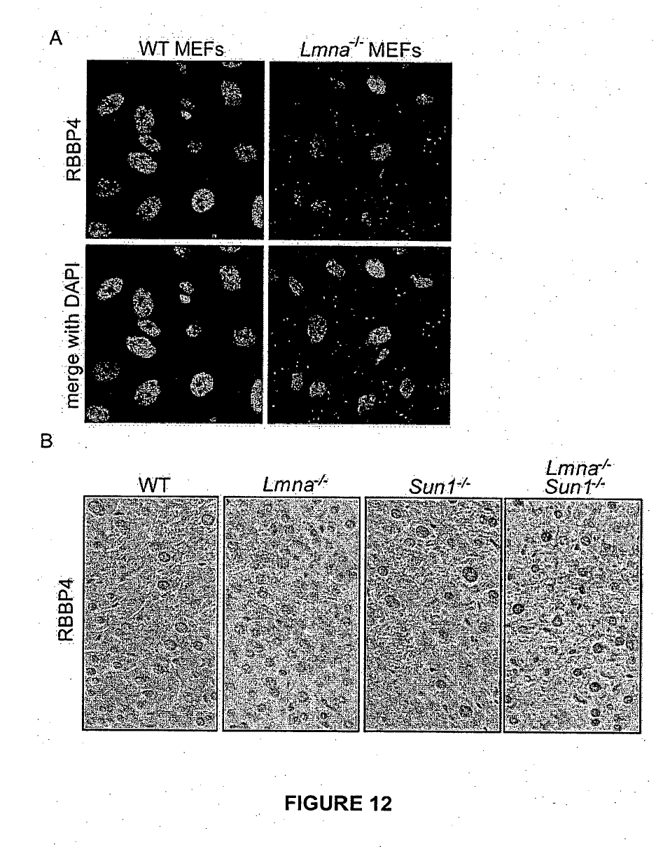

Chromatin disorganization and massive heterochromatin loss are correlated with nuclear shape alterations in HGPS cells. Assays for HGPS heterochromatin loss have included markers such as the lamin A-associated NURD (nucleosome remodeling and deacetylase) component RBBP4 and the pan heterochromatin marker histone H3K9me3. To corroborate the nuclear morphology findings (FIG. 9), the inventors investigated how SUN1 expression correlates with heterochromatin changes previously described for HGPS. When HGPS or normal skin fibroblasts were stained for RBBP4 (FIG. 11A, left) or H3K9me3 (FIG. 11A, right), an inverse correlation was observed between the expression of SUN1 and RBBP4 (FIG. 11B, left) or H3K9me3 (FIG. 11B, right). In agreement with the results in FIG. 9A, only a subset of HGPS cells were bright-SUN1 staining (yellow arrows=bright-SUN1, white arrows=dim-SUN1, FIG. 11A); and interestingly the bright-SUN1 cells were also those with the larger more distorted nuclei as well as staining sparsely for RBBP4 (FIG. 11A, B, left) or H3K9me3 (FIG. 11A, B, right). Separately, we found that RBBP4 expression was substantially reduced in ˜70% of Lmna^{-/-} MEFs (FIG. 12A) and in Lmna^{-/-} mouse liver tissue (FIG. 12B), further supporting an inverse relationship between Sun1 and NURD activity.

The inventors next asked if siRNA knock down of SUN1 would reverse HGPS-associated heterochromatin changes. A control-RNAi and SUN1-RNAi transfected HGPS (AG03513) cells were compared and surprisingly the inventors found that the latter cells did recover RBBP4 expression relative to the former (FIG. 11C). Because heterochromatin dysregulation is correlated with cellular senescence, and because HGPS cells exhibit premature senescence, the inventors queried how SUN1 affects HGPS senescence. To address this, the imventors knocked down SUN1 for 96 hours and examined acidic senescence associated β-galactosidase (SA-β-Gal) in control (AG03257) and HGPS (AG11498) cells (FIG. 11D). In normal cells, the extent of senescence was similar (˜9%) between control-siRNA or SUN1-siRNA treated samples (FIG. 11D); however, in HGPS cells, the observed high level of ambient senescence (˜22%) as measured by β-galactosidase was dramatically decreased (to ˜6%) after SUN1 knock down. Moreover, HGPS fibroblasts when treated with SUN1-RNAi gained a proliferative advantage over control-RNAi treated cells (FIG. 11E). These data collectively support the interpretation that increasing SUN1 accumulation is associated with HGPS pathology and removing over-expressed SUN1 restores normal cellular physiology.

Surprisingly, the inventors show that aberrant Sun1 expression is a critical pathogenic event common to Lmna^{-/-}, LmnaΔ9, and HGPS disorders. No other studies previously demonstrate this result. As noted here and elsewhere, Lmna^{-/-} mice, LmnaΔ9 mice, and HGPS individuals share a constellation of disorders that include nuclear aberrations, dystrophic organ and tissue abnormalities, and abbreviated lifespan. A current view is that progerin is causal of the LAΔ50 HGPS disease. How progerin mechanistically signals cellular and tissue damage remains elusive. That said, the existence of the dystrophic and cardiomyopathic pathologies in Lmna^{-/-} mice and multiple examples of Lmna mutations that do not synthesize progerin, but do produce degenerative-dystrophic diseases such as Emery-Dreifuss muscular dystrophy, Charcot-Marie-Tooth, Mandibuloacral dysplasia, Dunnigan-type familial partial lipdystrophy, atypical Werner's syndrome and limb girdle muscular dystrophy, requires an understanding of progerin-independent and dependent factors/cofactors underlying the pathologies.

The inner nuclear envelope Sun1 protein connects nucleoplasm with the cytoskeleton. Sun1 has various roles in nuclear anchorage, nuclear migration, and cell polarity, and deficits in Sun1 correlate with developmental retardation in neurogenesis, gametogenesis, myogenesis, and retinogenesis. However, to date, how an inner nuclear envelope protein like Sun1 fits into the pathogenesis of laminopathies is unknown.

The major unexpected finding here is that while Lmna^{-/-} mice and LmnaΔ9 mice thrive poorly and die prematurely, the removal of Sun1 creating Lmna^{-/-}Sun1^{-/-} and LmnaΔ9Sun1^{-/-} mice rescued pathologies and dramatically improved longevity (FIGS. 1, 2). To better understand these results, we observed that at the cellular level Lmna^{-/-} and LmnaΔ9 fibroblasts had uniformly increased Sun1 expression with significant protein mis-accumulation in the Golgi (FIG. 3A and FIG. 4).

Furthermore, approximately one in three LAΔ50 HGPS fibroblasts (FIGS. 9, 10, 11, and 13) was elevated for SUN1 expression with the bright (high)-SUN1, but not the dim (low)-SUN1, cells also exhibiting abnormal nuclear size and shape, heterochromatin RBBP4 and H3K9me3 markers, and cellular senescence (FIG. 9, 11). While one cannot do a Sun1 knock out experiment in LAΔ50 HGPS individuals, the knock down of SUN1 in LAΔ50 HGPS cells considerably improved nuclear size/shape defects, heterochromatin loss, and cellular senescence (FIG. 9, 11). Thus, while the approaches (knock out and knock down) and disease models (Lmna^{-/-}, LmnaΔ9, and LAΔ50 HGPS) are not identical, and one may suggest, and as, a parsimonious interpretation, consistent with the collective results is that Sun1 over accumulation represents a common effector of Lmna^{-/-}, LmnaΔ9, and LAΔ50 HGPS pathologies.

Based on these findings, the present invention provides Sun1 inhibitor for the treatment of laminopathies. Sun1 is normally located in the NE, in part positioned there by direct or perhaps indirect interactions with the lamin A filaments underlying the nuclear matrix. As noted above, a SUN1 protein deleted in its N-terminal (˜100 amino acids) lamin A-interacting domain relocates from the NE to the Golgi [FIG. 7]. Emerging evidence suggests that the SUN1-related SUN2 protein has a Golgi-retrieval sequence, which is required for retrieval of SUN2 from the Golgi to the ER. Differences between the two proteins may explain why Sun1, but not Sun2, expressed in the absence of cell endogenous lamin A (i.e. Lmna^{-/-} cells; FIG. 3, FIG. 4) accumulates in the Golgi. Several lines of investigation show that Sun1 accumulation arises from reduced protein turnover (FIG. 5C) and not increased transcription (FIG. 4F, 10C), suggesting that approaches to enhance protein degradation might be therapeutically beneficial.

The inventors unexpectedly found Golgi-storage of Sun1 is cytotoxic. Golgi targeting experiments with mSun1-Tgn38 (FIG. 6) and SUN1 103-785 mutant protein (FIG. 7) illustrated that. This toxicity may be akin to that elicited in abnormal human lysosomal- or ER-storage diseases. Aside from organelle storage disorders, other types of protein aggregation maladies like Alzheimer's have also been described. In Alzheimer's disease, evidence now suggests that it is the small soluble amyloid-β oligomers, not the large easily visualized amyloid-β fibrils/plaques, which result in neurotoxicity. As mentioned above, the inventors currently do not exclude that Golgi accumulation of SUN1 may indeed occur in LAΔ50 HGPS cells in vivo and that such cells may have rapidly succumbed and therefore are not represented in the mostly late passage repository-deposited HGPS fibroblasts (FIG. 13) available for our experiments. However, like soluble amyloid-β oligomers which need not present as gross aggregates to be cytotoxic, it may be that the degree of SUN1 over expression in LAΔ50 HGPS cells (FIG. 9E) is sufficient to functionally trigger pathology without having to reach levels required for overt Golgi-spillage.

Progerin underlies LAΔ50 HGPS disease development. In primary LAΔ50 HGPS cells or LmnaΔ9 mice where progerin (FIG. 10B) or lamin A-ΔExon9 protein is expressed, Sun1 knock down is sufficient to remedy cellular aberrancies, and senescence and longevity defects (FIGS. 1, 9, 11). A cogent interpretation of these results is that SUN1 accumulation is positioned downstream of progerin or lamin A-ΔExon9 such that the depletion of SUN1 sufficiently interrupts pathologic signaling. In Lmna^{-/-} mice where no progerin protein is synthesized, our data show that Sun1 accumulation remains pivotal to the cause of loss-of-lamin A disease. The present invention suggest that at least in the Lmna^{-/-}, LmnaΔ9, and LAΔ50 HGPS diseases, Sun1 over accumulation is critical to pathogenis. If this notion can be broadly applied, it then suggests that future clinical trials and therapies for laminopathies, which treat disease upstream events (i.e. targeting progerin) without resolving downstream pathogenic events (i.e. Sun1 misaccumulation) may be ineffective.

### EXPERIMENTAL METHODS

### Animals

Knockout mice were created using standard procedures. Because both Sun1^{-/-} and Lmna^{-/-} mice are reproductively defective, Sun1^{+/-} mice were crossed with Lmna^{+/-} mice to generate Lmna^{-/-}Sun1^{-/-} mice or Sun1^{+/-} mice were crossed with Lmna^{L530P/+} mice to generate LmnaΔ9Sun1^{-/-} mice. Mouse genotypes were verified by PCR. All animal experiments were conducted according to animal study protocols approved by the NIH Animal Use Committee or the Singapore Animal Use Committee.

Immunofluorescence and Confocal Microscopy

Cells were fixed in 4% paraformaldehyde in PBS for 30 minutes and permeabilized with 0.1% TritonX-100 for 5 minutes at room temperature. Cells were incubated with 1% BSA in PBS for 30 minutes to block nonspecific binding. Antibodies were added at dilutions of 1:100 to 1:1000 and incubated for 1.5 hours at room temperature. After three washes with PBS, cells were probed with fluorescent (Alexa-488, Alexa-594 or Alexa-647)-conjugated secondary antibodies. Nuclei were counterstained with Hoechst33342 or DAPI (Invitrogen). Cells were mounted onto glass slides with ProLong Gold antifade reagents (Invitrogen), and were visualized using a Leica TCS SP5 confocal microscope. Immunofluorescence intensity of Sun1 was quantified by the ImageJ 1.42q software (NIH) or by MetaMorph (Molecular Devices).

Cell Culture

Normal (AG03512, AG03257, AG03258, AG08469) and HGPS (AG01972, AG06297, AG11498, AG11513, AG06917, AG03513, AG03198) human skin fibroblasts were from the National Institute of Aging (NIA) Aged Cell Repository distributed by the Coriell Institute. Cells were maintained in high glucose MEM containing 10%-15% FBS and supplemented with 2 mM L-glutamine, 1 mM sodium pyruvate and antibiotics. Mouse embryonic fibroblasts (MEFs) were prepared from E15.5 embryos. Cells were dissociated by trypsin and were maintained in Dulbecco's modified eagle medium (DMEM) supplemented with 15% fetal bovine serum (FBS), 2 mM L-glutamine and antibiotics.

Plasmids

The mouse Sun1 (mSun1, accession number: NM_{—}024451, 913 a. a.), mSun1-FLAG, mSun1-Tgn38-HA, full length human SUN1 (hSUN1-HA, accession number: NM_{—}001130965, 785 aa), hSUN1 (aa 103-785)-HA and mouse lamin A expression plasmids were constructed based on the pcDNA3.1 vector (Invitrogen). All the constructs generated were verified by DNA sequencing, and the expression of the cloned genes was confirmed by western analyses. Lipofectamine 2000 (Invitrogen) and PolyJet (SignaGen Laboratories) were used for plasmid transfections.

Reagents, Primers, and RT-PCR

Reagents were obtained from the following resources. Sigma-Aldrich: nocodazole (M1404), lactacystin (L6785), brefeldin A (BFA, B5936), latrunculin B (LAT-B, L5288), cycloheximide (C4859). Primer sequences for Sun1 genotyping: 5′-GGCAAGTGGATCTCTTGTGAATTCTTGAC-3′ and 5′-GTAGCACCCACCTTGGTGAGCTGGTAC-3′.

WT mice produced a 1262 bp fragment and the Sun1 knockout mice produced a 263 bp fragment. Primer sequences for Lmna genotyping: common forward primer for WT and Lmna KO 5′-AGTTCGTGCGGCTGCGCAACAAGTCCAACG-3′; reverse primer for WT: 5′-GTCATCAAAGGATCGTCACCATTCTGAC-3′; reverse primer for Lmna KO: 5′-CCATTCGACCACCAAGCGAAACATCGC-3′. Wild-type mice produced a 500 bp fragment and the Lmna knockout mice produced an 850 bp fragment.

For RT-PCR, total RNA was extracted from MEFs using TRIzol (Invitrogen). Complementary DNA (cDNA) was produced from MEFs RNA (5 mg) using the SuperScript II Reverse Transcriptase Kit (Invitrogen). Three pairs of primer p177/p178 (p177: 5′-GGGACAGCCAGGCTATTGATT; p178: 5′-CATGGCTTGTGCTCGAGGA), p1213/p1379 (p1213: 5′-CTTCTTACCAGGTGCCTTCG; p1379:5′-GAATCGTCCACCCTCTGTGT), and p140/p141 (p140: 5′-TATTGTGTCTGCCGTGAATC; p141: 5′-GCCGTCTTGGTCTCATAGGTC) were used to amplify three coding regions of mouse Sun1, respectively. PCR products of mouse glyceraldehyde-3-phosphate dehydrogenase (Gapdh-F: 5′-TCACCACCATGGAGAAGGC; Gapdh-R: 5′-GCTAAGCAGTTGGTGGTGCA) were served as an internal control. Primers for RT-PCR of human SUN1 (hsSUN1-F: 5′-GGACGTGTTTAAACCCACGACTTCTCG; hsSUN1-R: 5′-CTCTGACTTTAGCTGATCCAGCTCCAGC), human GAPDH (GAPDH-F: 5′-AGCCACATCGCTCAGACACC;GAPDH-R: 5′-GTACTCAGCGGCCAGCATCG).

Antibodies

The rabbit anti-SUN domain of mouse Sun1 (aa 701-913) was prepared as described in the art. Specificity of this antibody in western blot and immunofluorescence staining was examined and verified by comparing the signals from wild-type and Sun1^{-/-} MEFs. The rabbit anti-human SUN1 antibody was prepared as described previously. Other antibodies were obtained from the following resources. Abcam: rabbit anti-GM130 (ab52649), rabbit anti-H3K9me3 (ab8898), rabbit anti-Sun2 (ab87036), mouse anti-RBBP4 (ab488); Sigma-Aldrich: mouse anti-atubulin (T5168), mouse anti-Actin (A1978), mouse anti-HA (H3663), mouse anti-FLAG (F1804), rabbit anti-FLAG (F7425), rabbit anti-GM130 (G7295); Santa Cruz Biotechnology: mouse anti-lamin A/C (sc-7292), goat anti-lamin B1 (sc-6217), rabbit anti-Emerin (sc-15378); Covance: mouse anti-Nup153 (MMS-102P), mouse anti-human SUN1 (customized); Epitomics: rabbit anti-RBBP4 (2599-1). BD Transduction Laboratories: mouse anti-Calnexin (610524); mouse anti-GM130 (610823).

Western Blotting

To extract nuclear envelope proteins from human skin fibroblasts, cultured cells were washed twice with PBS. The cell pellet was incubated with ice-cold RIPA buffer [50 mM HEPES, pH 7.3, 150 mM NaCl, 2 mM EDTA, 20 mM β-gylcerophosphate, 0.1 mM Na₃VO₄, 1 mM NaF, 0.5 mM DTT and protease inhibitor cocktail (Roche)] containing 1% NP-40 and 1% SDS plus mild sonication. Lysates were then analyzed by 8% SDS-PAGE, transferred to polyvinylidene fluoride (PVDF, Millipore) membrane and blotted antibodies. Corresponding alkaline phosphatase-conjugated secondary antibodies (Sigma-Aldrich) were added, and the blots were developed by chemiluminescence following the manufacturer's protocol (Chemicon).

RNAi

Synthetic Stealth siRNA duplexes targeting human SUN1 (5′-CCAUCCUGAGUAUACCUGUCUGUAU-3′) were from Invitrogen. Small interfering RNAs were induced into human skin fibroblasts using the Lipofectamine 2000 transfection reagent (Invitrogen) or Lipofectamine RNAiMax trasnfection reagent (Invitrogen). For siRNA delivery using Lipofectamine 2000, 60 pmol of siRNA mixed with 3 ml of Lipofectamine 2000 transfection reagent were used per well in a 12-well plate. For Lipofectamine RNAimax for siRNA delivery, only 3 pmol and 2 ml of the transfection reagent were used per well in a 12-well plate.

SiRNA Transfection of HeLa Cells

HeLa Cells were seeded in to 6 well tissue culture plates containing glass coverslips. The cells were incubated at 37° C. and in a humidified atmosphere containing 5% CO2 until 50% confluent. For each well, the following oligonucleotide transfection conditions were employed: (A) 10 μl of a 20 μM stock solution of oligonucleotide was mixed with 175 μl of Opti-MEM Reduced Serum Medium (Invitrogen) in a sterile 1.5 ml tube; (B) In a separate tube 3 μl of Oligofectamine Transfection Reagent (Invitrogen) was combined with 12 μl of Opti-MEM to give a final concentration of 15 μl; (C) The contents of both tubes (A and B) were then combined and incubated at room temperature for 20 min; (D) The normal medium was removed from the cells and replaced with 800 μl of serum-free medium (Dulbecco's MEM) and the 200 μl of the combined Oligonucleotide-Oligofectamine mix (C). The cells were then returned to the incubator; (E) after 4 h, 350 μl of DMEM combined with 150 μl of foetal calf serum was added to the cells. These were returned to the incubator for 48-72 h; (F) The cells were then processed for immunoflorescence microscopy using conventional procedures and employing an anti-Sun1 antibody.

Golgi Fractionation

Golgi fractionation was performed using the Golgi isolation kit (Sigma-Aldrich, GL0010) according to the manufacturer's protocol with some modifications. Mouse liver was minced with 1 ml of 0.25 M sucrose isolation solution per 1 g of tissue. The tissue suspension was homogenized with six slow motions of the PTFE pestle at 300 rpm and centrifuged at 3,000×g for 15 min at 4° C. Supernatant was transferred to a fresh tube and concentration of sucrose was adjusted to 1.25 M. A discontinuous gradient was built in an ultracentrifuge tube by adding 1.84 M sucrose solution, the sample (sucrose concentration adjusted to 1.25 M), 1.1 M sucrose solution and 0.25M sucrose solution sequentially. After centrifugation at 12,000×g for 3 hr, the Golgi-enriched fraction from the 1.1 M/0.25M sucrose interphase was withdrawn and subjected to western analyses.

Senescence Assay

The senescence associated β-galactosidase (SA-β-Gal) assay was performed by following protocol of the Cellular Senescence Assay Kit from Cell Biolabs, Inc.

Cell Proliferation Assay

Cell proliferation was performed by quantifying viable cells with Cell Counting Kit-8 (Fluka) according to the manufacturer's protocol.

Micro-CT

Wild-type, Sun1^{-/-}, Lmna^{-/-} and Lmna^{-/-}Sun1^{-/-} mice were examined by compact cone-beam tomography (MicroCAT-II scanner). Whole-body scans were performed in the axial plane with the specimens mounted in a cylindrical sample holder. Micro-computed tomography (micro-CT) was performed at 55 kVp, with an anode current of 500 mA and a shutter speed of 500 ms. The femur bone specimens were fixed in 10% formalin buffered with phosphate and then examined by SkyScan 1172 Micro-CT.

Three-dimensional images of the skeletons were reconstructed from the micro-CT scanning slices and used for analyses of the skeletal structure and morphology. Quantitative data were calculated by SkyScan CT-analyzer Software Guide. A manufacturer-provided hydroxyapatite phantom of known density was used to calibrate the mean density of bone volume and the cortical thickness.

MRI

Mouse cardiac magnetic resonance imaging (MRI) was conducted by following the NIH animal care and use guidelines. MRI experiments were performed in a 7.0T, 16-cm horizontal Bruker MR imaging system (Bruker) equipped with Bruker ParaVision 4.0 software. Mice were anesthesized with 1.5%-3% isoflurane and imaged with ECG, temperature and respiratory detection using a 38 mm Bruker birdcage volume coil. Magnevist (gadopentate dimeglumine contrast agent, Bayer HealthCare) diluted 1:10 with sterile 0.9% saline, was administered subcutaneously at 0.3 mmol Gd/kg. Intravenous route was not used due to small size of some mice (ca. 10-12 g) with invisible tail veins. Ti weighted gradient echo cine images of the heart were acquired in short axis from above the base to the apex (6-10 slices depending on slice thickness) with the following parameters: repetition time TR=11 ms, echo time TE=3.5 ms, 11 to 14 frames, 30 degree flip angle, 2.8 to 3.0 cm field of view, 256×256 matrix, respiratory and ECG-gated. 1.0 mm slice thickness with 4-5 averages was used on mice over 12 g and 0.75 mm thickness with 4-7 averages for mice less than 12 g. Cardiac MRI data were processed to determine ejection fractions and associated functional parameters using the CAAS-MRV-FARM software (Pie Medical Imaging, Netherlands.)

Statistics

Means and standard deviation are presented to describe the distribution. Student t test was used to compare mean difference between two groups. ANOVA analysis was performed to compare mean difference among groups. Multiple comparisons were carried out by Scheffe's Test. Kaplan-Meier method was used to draw the survival curves. Log-rank test was conducted on the homogeneity of survival curves among four types of mouse. We used Mixed model to compare the difference between body weight during the followed period among four types of mouse. We also used Generalized Estimating Equations (GEE) Method to compare the cell number among four types of MEF cells. The working correlation structure was set unstructured, and the linked function was set Poission distribution. Statistics were carried out by SAS 9.2 or GraphPad Prism 5.0.

1. A Sun1 inhibitor for use in treating a laminopathy.

2. The Sun1 inhibitor according to claim 1, wherein the Sun1 inhibitor is selected from the group consisting of a silencing oligonucleotide, a ribozyme, a Transcription Activator-Like Effector Nuclease (TALEN) and a Zinc Finger Nuclease (ZFN).

3. The Sun1 inhibitor according to claim 2, wherein the silencing oligonucleotide is selected from the group consisting of a small interfering RNA (siRNA), a short hairpin RNA (shRNA), a morpholino oligomer, and a microRNA (miRNA) mimic.

4. The Sun1 inhibitor according to claim 3, wherein the siRNA has a sequence selected from the group consisting of SEQ ID NOs: 1 to 47 or a variant thereof.

5. The Sun1 inhibitor according to claim 2, wherein the silencing oligonucleotide comprises a chemical modification of one or more nucleotides, which render the silencing oligonucleotide more stable than the non-modified sequence.

6. The Sun1 inhibitor according to claim 5, wherein the modification comprises a modification of the phosphate backbone, a modified sugar moiety, a modified nucleotide, or a modified terminal nucleotide.

7. The Sun1 inhibitor according to claim 6, wherein the modified sugar moiety is selected from the group consisting of 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine or 2′-amino-butyryl-pyrene-uridine.

8. The Sun1 inhibitor according to claim 6, wherein the modification of the phosphate backbone comprises replacing one or more or all of the phosphate molecules of the nucleotide phosphate backbone with a molecule selected from the group consisting of phosphorothioate, methylphosphonate, phosphotriester, phosphorodithioate and phosphoselenate.

9. The Sun1 inhibitor according to claim 6 wherein the modified terminal nucleotide has its 2′-OH group substituted with a molecule selected from the group consisting of alkyl, substituted alkyl, alkaryl-, aralkyl-, —F, —Cl, —Br, —CN, —CF₃, —OCF₃, —OCN, —O-alkyl, —S-alkyl, —O— allyl, —S-allyl, HS-alkyl-O, —O-alkenyl, —S-alkenyl, —N-alkenyl, —SO-alkyl, -alkyl-OSH, -alkyl-OH, —O-alkyl-OH, —O-alkyl-SH, —S-alkyl-OH, —S-alkyl-SH, -alkyl-S-alkyl, -alkyl-O-alkyl, —ONO₂, —NO₂, —N₃, —NH₂, alkylamino, dialkylamino-, aminoalkyl-, aminoalkoxy, aminoacid, aminoacyl-, —ONH₂, —O-aminoalkyl, —O-aminoacid, —O-aminoacyl, heterocycloalkyl-, heterocycloalkaryl-, aminoalkylamino-, polyalklylamino-, substituted silyl-, methoxyethyl-(MOE), alkenyl and alkynyl.

10. The Sun1 inhibitor according to claim 6, wherein the modified nucleotide comprises a modified base, wherein the modified base is selected from the group consisting of 2-aminoadenosine, 2,6-diaminopurine, inosine, pyridin-4-one, pyridin-2-one, phenyl, pseudouracil, 2,4,6-trimethoxy benzene, 3-methyl uracil, dihydrouridine, naphthyl, aminophenyl, 5-alkylcytidine (e.g., 5-methylcytidine), 5-alkyluridine (e.g., ribothymidine), 5-halouridine (e.g., 5-bromouridine), 6-azapyrimidine, 6-alkylpyrimidine (e.g. 6-methyluridine), propyne, queuosine, 2-thiouridine, 4-thiouridine, wybutosine, wybutoxosine, 4-acetylcytidine, 5-(carboxyhydroxymethyl)uridine, 5-carboxymethylaminomethyl-2-thiouridine, 5-carboxymethylaminomethyluridine, beta-D-galacto sylqueuo sine, 1-methyladenosine, 1-methylinosine, 2,2-dimethylguano sine, 3-methylcytidine, 2-methyladeno sine, 2-methylguano sine, N6-methyladeno sine, 7-methylguano sine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladeno sine, beta-D-mannosylqueuosine, uridine-5-oxyacetic acid, 2-thiocytidine, 3,N(4)-ethanocytosine, 8-hydroxy-N6-methyladenine, 4-acetylcytosine, 5-fluorouracil, 5-bromouracil, 5-carboxymethylaminomethyl-2-thiouracil, 5 carboxymethylaminomethyl uracil, dihydrouracil, N6-isopentyl-adenine, 1-methylpseudouracil, 1-methylguanine, 2,2-dimethylguanine, 2-methylguanine, 3-methylcytosine, N6-methyladenine, 5-methoxyaminomethyl-2-thiouracil, β-D-mannosylqueuosine, 5-methoxycarbonylmethyluracil, 2 methylthio-N6-isopentenyladenine, uracil-5-oxyacetic acid methyl ester, pseudouracil, 2-thiocytosine, 5-methyl-2 thiouracil, 2-thiouracil, 4-thiouracil, 5-methyluracil, N-uracil-5-oxyacetic acid methylester, uracil 5-oxyacetic acid, 2-thiocytosine, 5-propyluracil, 5-propylcytosine, 5-ethyluracil, 5-ethylcytosine, 5-butyluracil, 5-pentyluracil, 5-pentylcytosine, and 2,6,-diaminopurine, methylpseudouracil, 1-methylguanine and 1-methylcytosine.

11. The Sun1 inhibitor according to claim 2, wherein the silencing oligonucleotide is formulated with a delivery vehicle.

12. The Sun1 inhibitor according to claim 11, wherein the delivery vehicle is a nanoparticle selected from the group consisting of a liposome, a peptide, an aptamer, an antibody, a polyconjugate, a microencapsulation, a virus like particle (VLP), a nucleic acid complex, or a mixture thereof.

13. The Sun1 inhibitor according to claim 12, wherein the liposome is a stable nucleic acid-lipid particle (SNALP), or 1,2-dioleoyl-sn-glycero-3-phosphocholine (DOPC) based delivery system, or a lipoplex.

14. The Sun1 inhibitor according to claim 2, wherein the silencing oligonucleotide is formulated for systemic administration.

15. The Sun1 inhibitor of claim 1, wherein the laminopathy is selected from the group consisting of Hutchinson-Gilford Progeria syndrome (HGPS); Emery-Dreifuss Muscular Dystrophy (EDMD); cardiomyopathy; Atypical Werner syndrome; Barraquer-Simons syndrome; Buschke-Ollendorff syndrome; Charcot-Marie-Tooth disease; Familial partial lipodystrophy of the Dunnigan type (FPLD); Greenberg dysplasia; Leukodystrophy; Limb-girdle muscular dystrophy type 1B; Lipoatrophy with diabetes, hepatic steatosis, hypertrophic cardiomyopathy, and leukomelanodermic papules (LDHCP); Mandibuloacral dysplasia with type A lipodystrophy (MADA); Mandibuloacral dysplasia with type B lipodystrophy (MADB); Pelger-Huet anomaly (PHA); Pelizaeus-Merzbacher disease and Tight skin contracture syndrome

16. (canceled)

17. A method of treating a laminopathy comprising the administration of an effective amount of a Sun1 inhibitor according to claim 1 to a mammal in need thereof.

18. A siRNA having a sequence which is complementary to the Sun1 mRNA sequence.

19. The siRNA of claim 18, wherein the siRNA is 8 to 50 nucleotides long, or 10 to 50 nucleotides long, or 20 to 50 nucleotides long, or 30 to 50 nucleotides long, or 10 to 40 nucleotides long, or 10 to 30 nucleotides long, or 20 to 40 nucleotides long, or 30 to 40 nucleotides long.

20. The siRNA of claim 19, wherein the siRNA further comprises a chemical modification of one or more nucleotides as recited in claim 5.

21. An oligonucleotide having a sequence according to any one of SEQ ID NOs: 1 to 47.

22. The oligonucleotide of claim 21, wherein the oligonucleotide further comprises a chemical modification of one or more nucleotides as recited in claim 5.

23. A method of diagnosing a laminopathy, or determining if an individual is at risk of developing a laminopathy, comprising the steps of:
a. measuring the expression level of Sun1 in an individual or a sample obtained from the individual;
b. comparing the Sun1 expression levels obtained from step (a) with a control reference wherein an elevated level of Sun1 in the individual compared to the control indicates that the individual has a laminopathy or is at risk of developing a laminopathy.

24. A method of monitoring the progression or treatment of a laminopathy, comprising the steps of:
a. measuring the expression level of Sun1 in an individual or a sample obtained from the individual;
b. comparing the Sun1 expression levels obtained from step (a) with a control reference wherein an elevated level of Sun1 in the individual compared to the control indicates that the laminopathy has progressed from a less advanced stage to a more advanced stage.

25. The method of claim 23, wherein the laminopathy is selected from the group consisting of Hutchinson-Gilford Progeria syndrome (HGPS); Emery-Dreifuss Muscular Dystrophy (EDMD); cardiomyopathy; Atypical Werner syndrome; Barraquer-Simons syndrome; Buschke-Ollendorff syndrome; Charcot-Marie-Tooth disease; Familial partial lipodystrophy of the Dunnigan type (FPLD); Greenberg dysplasia; Leukodystrophy; Limb-girdle muscular dystrophy type 1B; Lipoatrophy with diabetes, hepatic steatosis, hypertrophic cardiomyopathy, and leukomelanodermic papules (LDHCP); Mandibuloacral dysplasia with type A lipodystrophy (MADA); Mandibuloacral dysplasia with type B lipodystrophy (MADB); Pelger-Huet anomaly (PHA); Pelizaeus-Merzbacher disease and Tight skin contracture syndrome.
